(19) United States
(12) Patent Application Publication
(10) Pub. No.: US 2024/0157166 A1
(43) Pub. Date: May 16, 2024

(54) ALTERNATING MAGNETIC FIELDS AND ANTIBIOTICS TO ERADICATE BIOFILM ON METAL IN A SYNERGISTIC FASHION

(71) Applicant: Board of Regents, The University of Texas System, Austin, TX (US)

(72) Inventors: David Greenberg, Coppell, TX (US); Rajiv Chopra, Dallas, TX (US)

(21) Appl. No.: 18/552,709

(22) PCT Filed: Mar. 31, 2022
(86) PCT No.: PCT/US22/22892

§ 371 Date: Sep. 27, 2023

Related U.S. Application Data
(60) Provisional application No. 63/325,298, filed on Mar. 30, 2022.
(60) Provisional application No. 63/169,636, filed on Apr. 1, 2021.

(51) Int. Cl. A61N 2/02; A61F 2/30; A61N 2/00

(52) U.S. Cl. CPC A61N 2/02; A61F 2/30; A61N 2/004; A61F 2002/30668; A61F 2002/30719

(57) ABSTRACT

Metal-associated infections such as prosthetic joint infection (PJI) cause significant morbidity across the world. Infected implants frequently require surgical removal and weeks of antibiotics. This is in large part due to the formation of biofilm. Embodiments described herein utilize alternating magnetic fields (AMF) as a non-invasive approach for eradicating (i.e., significantly reducing) biofilm off of metal. Embodiments apply brief intermittent bursts of AMF given in concert with traditional antibiotics to synergistically remove biofilm off of metal. This effect is seen across common PJI-associated pathogens and with clinically used antibiotics. Utilizing AMF in an intermittent fashion has important implications for providing a non-invasive treatment that could be both safe and effective in patients.

### CROSS-REFERENCE TO RELATED APPLICATIONS

This application claims priority to U.S. Provisional Patent Application No. 63/169,636 filed on Apr. 1, 2021 and entitled “Alternating Magnetic Fields and Antibiotics to Eradicate Biofilm on Metal in a Synergistic Fashion”, the content of which is hereby incorporated by reference. This application also claims priority to U.S. Provisional Patent Application No. 63/325,298 filed on Mar. 30, 2022 and entitled “Alternating Magnetic Fields and Antibiotics to Eradicate Biofilm on Metal in a Synergistic Fashion”, the content of which is hereby incorporated by reference.

This invention was made with government support under AI155291 awarded by The National Institutes of Health (NIH). The government has certain rights in the invention.

### BACKGROUND

An alternating magnetic field (AMF) is a non-invasive approach to treat implant associated infections.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1A, 1B, and 1C address simulation and measurements of intermittent alternating magnetic field (iAMF) heating. The experimental set-up consisted of a stainless-steel ring with biofilm in media in a 50-ml tube and held in place by a plastic holder (FIG. 1A). The tube is placed in a solenoid coil (simulated image in FIG. 1A). A representation of the iAMF dosing scheme (FIG. 1B) with doses separated by hours (Δtdose, panel top). Each dose is composed of multiple short-term exposures of AMF (Nexp) delivered at intervals (Δtexp) for several seconds of AMF heating (texp, the exposure time required to reach target temperature, Tmax) followed by temperature decline after AMF is shut off (FIG. 1B, middle image). Temperature versus time for the ring upon AMF exposure to a Tmax of 50, 65 and 80 degrees C. is shown (FIG. 1C). Simulated AMF heating of a metal ring for different exposure times depicts spatial temperature variation on the surface, and minimal heating of surrounding media. Mean and standard deviation of the temperature are shown.

FIGS. 2A, 2B, 2C, 2D address how iAMF and ciprofloxacin are synergistic in reducing P. aeruginosa biofilm. (FIG. 2A) The general treatment scheme for combining iAMF and antibiotics. (FIGS. 2B, 2C, 2D) Bacterial log reduction over a 24-hour period for PAO1 biofilm upon treatment with iAMF heating alone (blue dotted line), ciprofloxacin at 0.5 mg/mL alone (black solid line) or iAMF+ciprofloxacin (blue solid line) at different peak temperatures Tmax of (b) 80° C., (c) 65° C. or (d) 50° C. The number of exposures was varied for each case as shown in the panels. Untreated controls (black dotted line) were not exposed to antibiotics or AMF. Colony forming units (CFU) were counted at 0, 12 h (pre- and post-AMF) and 24 h post treatment. CFU limit of detection (LOD)=0.78 log(CFU/cm2). Statistical significance: not significant (ns) and significance at p<0.0001 (****).

FIGS. 3A, 3B, 3C, 3D address how iAMF and ciprofloxacin cause bacterial morphologic changes. Laser scanning confocal microscopy of P. aeruginosa (PAO1) biofilm-infected rings 12 h post start of treatment. Live bacteria within the biofilm express green fluorescent protein (GFP) while EPS are stained with ConcanavalinA-Alexa Fluor 647 conjugate, fluorescing red. Rings were (FIG. 3A) treated with iAMF (Tmax=65° C.) for 1 h, then incubated in MI-III media for 12 h, (FIG. 3B) incubated in ciprofloxacin at 0.5 μg/mL for 12 h or (FIG. 3C) treated with 1 h iAMF while incubating with 0.5 μg/mL of ciprofloxacin for 12 h. (FIG. 3D) Untreated control. Scale Bar: 100 μm.

FIG. 4 addresses how iAMF displays dose-dependent reductions of P. aeruginosa biofilm in combination with ciprofloxacin. iAMF doses (Tmax=65° C., Δtexp=5 min) were delivered at 0 and 12 h with 3, 6 or 12 exposures in each dose (incubated with 0.5 mg/mL of ciprofloxacin meanwhile). Colony forming units (CFU) were counted at 0, 12 and 24 h time points immediately after the first iAMF dose (left) and at 24 h post treatment (right). For treatment with ciprofloxacin alone (post first dose for no AMF) CFU was counted after 1 hour in ciprofloxacin. The CFU at time 0 was 6.81 log(CFU/cm²). CFU limit of detection (LOD)=0.78 log(CFU/cm²). p=0.0318 (*) and p<0.0001 (****).

FIGS. 5A and 5B address how iAMF and antibiotics are synergistic in reducing S. aureus biofilm. S. aureus (UAMS1) biofilm was treated with iAMF doses at 0 and 12 h (15 min/dose, Tmax=65° C., Δtexp=5 min) and specified antibiotic. (FIG. 5A) Biofilm log reduction (CFU) post 24 h with iAMF and 2 μg/mL ceftriaxone. CFU were counted at time points 0, 12 and 24 h. (FIG. 5B) CFU of S. aureus biofilm 24 h post-treatment with iAMF and ceftriaxone (2 μg/mL) or linezolid (2 μg/mL). CFU limit of detection (LOD)=0.78 log(CFU/cm2). Statistical significance: not significant (ns) p=0.0004 (***) and significance at p<0.0001 (****).

FIGS. 6A, 6B, and 6C address how iAMF can reduce MDR pathogens depending on the mechanism of resistance. MDR P. aeruginosa (MB699) biofilm was treated with meropenem (MIC 64 μg/mL) or ciprofloxacin (MIC 64 μg/mL) with or without iAMF (dosed at 0 and 24 h, Nexp=12, Tmax=65° C., Dtexp=5 min) while incubating with antibiotic for 48 h. FIG. 6A: Mechanism for sensitization of antibiotic-resistant biofilm to meropenem by AMF. FIG. 6B: Treatment time course with meropenem (left) or ciprofloxacin (right) at 64 μg/mL. Colony forming units (CFU) were counted at time points of 0, 24 and 48 h. FIG. 6C: Log reduction of antibiotic-resistant biofilm at different concentrations of ciprofloxacin or meropenem at 48 h post start of treatment. CFU limit of detection (LOD)=0.78 log(CFU/cm2). Statistical significance: not significant (ns), p=0.0001 (***), and p<0.0001 (****).

FIG. 7 addresses combined iAMF/antibiotic treatment of P. aeruginosa (PAO1) biofilm grown on plastic and metal rings with 0.5 μg/mL ciprofloxacin. Biofilm was treated with iAMF doses (dosing duration 1 h, Tmax=65° C., Δtexp=5 min) and 0.5 μg/mL ciprofloxacin at 0 h. Colony forming units (CFU) were counted at 12 h. CFU limit of detection (LOD)=0.78 log(CFU/cm2). Statistical significance: not significant (ns) and significance at p<0.0001 (****).

FIG. 8 addresses SEM images of antibiotic-resistant P. aeruginosa (MB699) biofilm treated with iAMF and antibiotics. Biofilm on a metal ring after treatment with iAMF (Nₑₓₚ=12, Tₘₐₓ=65° C., Δtₑₓₚ=5 min) and incubation in 64 μg/mL of meropenem or ciprofloxacin for 12 h. Magnification: 35,000×. Scale bar=300 nm.

FIG. 9 includes physical properties of materials used for simulation.

FIG. 10 includes iAMF parameters at different target temperature (Tmax). * 80 degrees C. was achieved within 6 s of exposure and was held near this temperature for an additional 6 s with a proportional-integral-derivative (PID) calibration before stopping AMF.

FIG. 11 includes minimum inhibitory concentrations of antibiotics used to treat strains of P. aeruginosa.

FIG. 12 includes a protocol or method in an embodiment.

FIG. 13 includes a method in an embodiment.

FIGS. 14, 15, and 16 include systems with which to implement embodiments.

FIG. 17 includes signal characteristics for burst exposures and signal characteristics for thermal exposures in embodiments.

FIG. 18 includes CEM43 measurements surrounding the ring during iAMF. With the assumption that the rings were surrounded by muscle tissue, a simulation was performed to calculate the CEM43 at different distances from the ring with iAMF. Three iAMF treatment conditions were used: Nexp=1, Tmax=80° C.; Nexp=1, Tmax=65° C.; Nexp=12, Tmax=65° C., Dtexp=5 min. 240 min indicated the threshold of non-reversible cellular damage.

FIG. 19 includes an FIC index of thermal treatment time and ciprofloxacin concentrations for biofilms. PAO1 biofilms were treated at 65° C. at time 0 for certain time periods, and incubated with ciprofloxacin at various concentrations for 12 h or 24 h at 37° C. The numbers in the heat map showed the FIC index values for the treatment combination. FIC values of less than or equal to 0.5 were considered to be a synergistic effect, values of >0.5 and <4 indicated no interaction or additivity, and values of greater than or equal to 4 indicated antagonistic effect. n=3.

FIGS. 20A, 20B, 20C, 20D show iAMF and antibiotics can work on biofilm of various ages. P. aeruginosa (PAO1) and S. aureus (UAMS1) biofilms were cultured until day 7 following the same protocol with media replenishment every 24 h. Then the biofilms were treated with iAMF doses at 0 and 12 h (Tmax=65° C., Δtexp=5 min, 15 min per dose) and specified antibiotics. (FIG. 20A) 7-day P. aeruginosa (PAO1) biofilm log reduction (CFU) post 24 h with iAMF and 0.5 μg mL-1 ciprofloxacin. CFU was counted at time points 0, 12 (pre- and post-AMF), and 24 h. (FIG. 20B) 7-day S. aureus (UAMS1) biofilm log reduction (CFU) post 24 h with iAMF and 2 μg mL-1 linezolid. CFU was counted at time points 0, 12, and 24 h. (FIG. 20C) Comparison of prior 2-day (48 h) biofilm and 7-day P. aeruginosa (PAO1) biofilm under the same iAMF (Tmax=65° C., Δtexp=5 min, 15 min per dose) treatment and 0.5 μg mL-1 ciprofloxacin at time 0 and 24 h. (FIG. 20D) Comparison of 2-day (48 h) biofilm and 7-day S. aureus (UAMS1) biofilm under the same iAMF (Tmax=65° C., Δtexp=5 min, 15 min per dose) treatment and 2 μg mL-1 linezolid at time 0 and 24 h. n=3. Error bars indicate SD. CFU limit of detection (LOD)=0.78 log(CFU cm-2). Two-way ANOVA. Statistical significance: not significant (ns).

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

In the previously filed provisional patent application reference was made to Exhibit A. Subject matter from Exhibit A is now included directly into this specification. Following the subject matter from Exhibit A is a discussion entitled “FURTHER DISCUSSION OF EMBODIMENTS”. References addressed in the following discussion of Exhibit A are located at the end of the specification's written description.

### Exhibit A

Embodiments address non-invasive intermittent alternating magnetic fields combined with antibiotics to reduce metal-associated biofilm in a synergistic fashion.

Hundreds of thousands of human implant procedures require surgical revision each year due to infection. Infections are difficult to treat with conventional antibiotics due to the formation of biofilm on the implant surface. Embodiments addressed herein include a non-invasive method to eliminate biofilm on metal implants using intermittent alternating magnetic fields (iAMF). As used herein, “eliminate” does not necessarily imply complete 100% removal or destruction of, for example, biofilm but can instead mean a significant reduction of, for example, biofilm. Embodiments demonstrate that iAMF and antibiotics are synergistic in their biofilm reducing capability. For Pseudomonas aeruginosa biofilm, bacterial burden was reduced >3 log with iAMF and ciprofloxacin after 24 hours compared with either treatment alone (p<0.0001). This effect was not limited by pathogen or antibiotic as similar biofilm reductions were seen with iAMF and either linezolid or ceftriaxone in Staphylococcus aureus. iAMF and antibiotic efficacy was seen across various iAMF settings, including different iAMF target temperatures, dose durations, and dosing intervals. Initial mechanistic studies revealed membrane disruption as one factor important for AMF enhanced antibacterial activity in the biofilm setting. Embodiments demonstrate the efficacy of utilizing a non-invasive approach to reduce biofilm off of metallic implants.

### Exhibit A Introduction

Metal implants such as prosthetic joints, bone fixation hardware, and dental implants, are widely used in medicine to replace damaged or diseased tissue (Reference 1). In aggregate, millions of metal devices are implanted into humans every year globally (Reference 2). In the case of total knee arthroplasty (TKA), over one million procedures are performed in the US each year, and the number is projected to reach ˜3.5 million by year 2030 due to population and health trends (Reference 3). Approximately 1-2% of these implants become infected. This serious complication is challenging to treat (Reference 3). Currently, treatment of prosthetic joint infections (PJI) mainly relies on multiple revision surgeries. An initial surgery is performed to remove the infected implant and a temporary spacer is placed (Reference 4). Antibiotics are administered for several weeks to clear residual infection. Once the patient is confirmed to be free of infection, a final surgery is performed to implant a new prosthesis (Reference 5). Treatment of PJI is highly invasive with a significant negative impact on patients' quality of life. Moreover, the failure rate of these multistage surgeries is currently over 10% (References 6, 7). In addition, the projected cost of treating PJI is 1.6 billion USD in 2020 in the United States alone, creating a significant economic burden to the health care system (Reference 8).

A primary reason that antibiotic treatment of metal implant infections (such as PJI) is ineffective is due to the formation of biofilm on the implant surface (Reference 9). Biofilm is a thin (tens to hundreds of micrometers) aggregate of bacteria and extracellular polymeric substances (EPS) (Reference 10). EPS is generated by bacteria and forms a barrier to the surrounding environment, rendering these organisms up to a thousand-fold more resistant to antibiotics as well as providing protection from the immune system (Reference 11). Importantly, increasing antibiotic resistance only further complicates this problem. Aside from PJI, biofilm also plays important roles in the infection of other widely used medical implants, including catheters, mechanical heart valves, and bone fixation hardware (Reference 1, 12, 13).

Non-surgical means of eradicating (i.e., significantly reducing) biofilm would be a significant advance in the treatment of metal implant infections (MII). Several physical approaches for eliminating (i.e., significantly reducing) biofilm have been proposed including electrical current (References 14-16), ultrasound (Reference 17), heat (References 18-20), and shock waves (Reference 21). However, these methods are either hard to apply in vivo or have limitations for use on metal implants. A potentially safer and more effective method of biofilm removal off of metal implants is through the use of AMF. AMF can be delivered from outside the body and does not suffer from penetration depth limitations or complex wave distortions through tissue boundaries. When metal implants are exposed to AMF, electrical currents are induced on the surface, resulting in the generation of heat. Previous studies have shown the feasibility and effectiveness of biofilm elimination (e.g., significant reduction) by AMF (References 19, 22). After just a few minutes of AMF treatment, the biofilm on a stainless-steel washer was reduced significantly (Reference 22).

However, Applicant determined the necessity to sustain temperatures ranging from 50-80 degrees C. for several minutes to achieve biofilm reduction presents challenges for AMF to be utilized clinically. In addition, incomplete eradication of bacteria via AMF results in regrowth within a short period of time (Reference 22). Embodiments include one approach to overcoming this obstacle, namely combination therapy with antibiotics. In vitro studies have demonstrated a greater and sustained reduction in bacterial burden. As such, Applicant determined AMF and ciprofloxacin in combination were observed to be more effective than AMF or ciprofloxacin alone in reducing biofilm and prevented its recurrence for up to 24 hours post treatment (References 20, 23, 24). In addition, Applicant noted utilizing brief, intermittent AMF exposures could address the issue of elevated implant temperatures and safety. As shown previously in a murine model, Applicant noted elevating a metal implant to a target temperature quickly and for a brief period resulted in much less tissue injury compared to longer duration exposures (Reference 25). Further, Applicant noted these short duration exposures can be delivered repeatedly with sufficient cool-down time in between exposures to allow for thermal doses that are therapeutic on the implant surface without a concomitant rise in tissue thermal dose. This approach is referred to as intermittent AMF, or iAMF.

Embodiments include the efficacy of iAMF exposures in combination with antibiotics to eliminate (i.e., significantly reduce) biofilm on metal surfaces in vitro. Applicants determines the relationship between AMF parameters (temperature, duration, # of exposures) and antibiotics (drug, concentration, dosing). Applicants explored this approach in both prototypic Gram-positive and Gram-negative pathogens and explore the mechanisms that underlie this mechanistic relationship by attempting to reduce multidrug-resistant pathogens with iAMF.

### Exhibit A Results

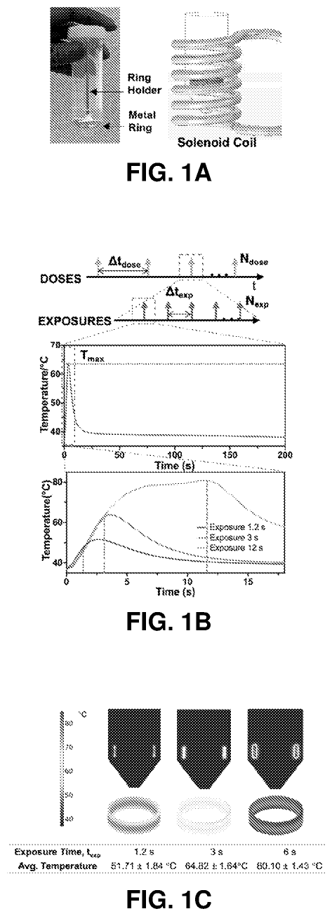

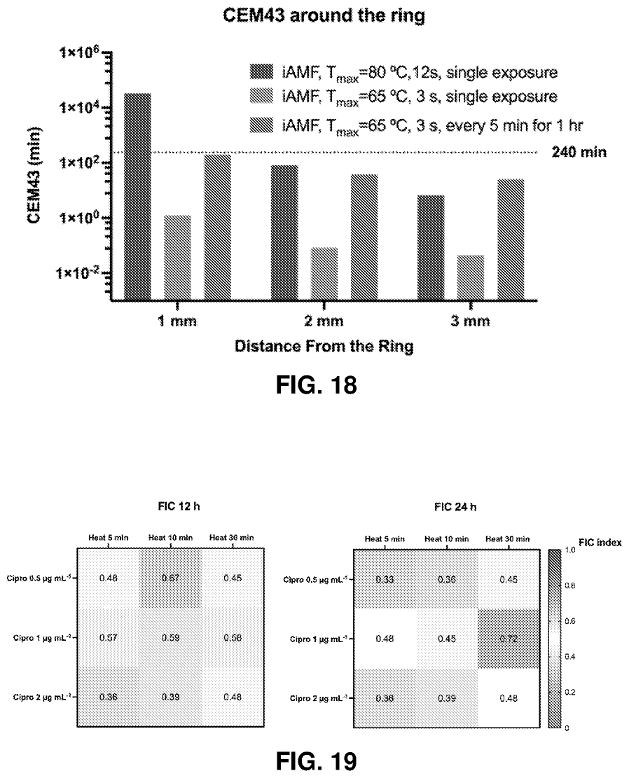

iAMF exposures were produced using an in vitro system designed to heat metal rings with precisely controlled exposure durations, and with specified exposure and dosing intervals. The system is comprised of 32 identical solenoid coils, capable of generating a uniform AMF (10.2±0.3 mT) at the center of each coil. In addition, the measured magnetic field agreed well with the predictions from simulation (11.2±0.4 mT). Metal rings were chosen since they were expected to heat uniformly in the magnetic field of a solenoid when oriented along the axis of the coil as shown in FIG. 1A. The Finite-element simulation results in FIG. 1C confirm the uniform heating achieved. The surface temperature distribution on the rings after 1.2, 3 and 6 s of heating are shown, with uniform temperatures around the circumference of the ring, and a standard deviation of no more than 2 degrees C. between the top and middle. Further, the simulations highlight that for these short durations of heating, the media surrounding the rings is not significantly heated, which was also observed by actual measurement. Cumulative equivalent minutes at 43° C. (CEM43) are used for evaluating mammalian cell thermal damage (Reference 26). Usually, 240 min is considered as the threshold for permanent damage in muscle tissue (References 27,28). Because the heat transfer from the rings to the adjacent media is governed by heat conduction and convection, Applicants calculated the CEM43 around the ring with the assumption that the ring was surrounded by muscle tissue (i.e., only heat conduction). The CEM43 did not exceed 240 min at 2 mm from the ring under iAMF with Tmax=80° C. and at 1 mm under 12 iAMF exposures of Tmax=65° C., suggesting no permanent tissue damage at this distance (FIG. 18).

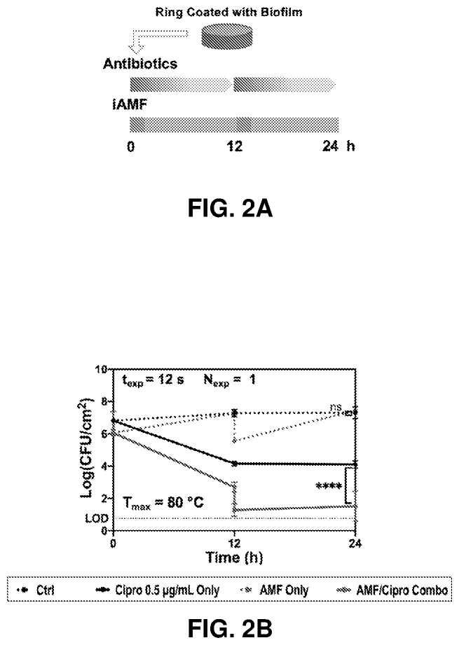

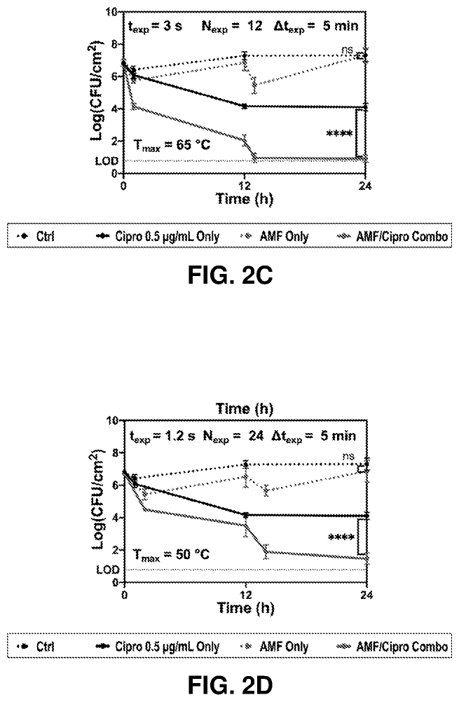

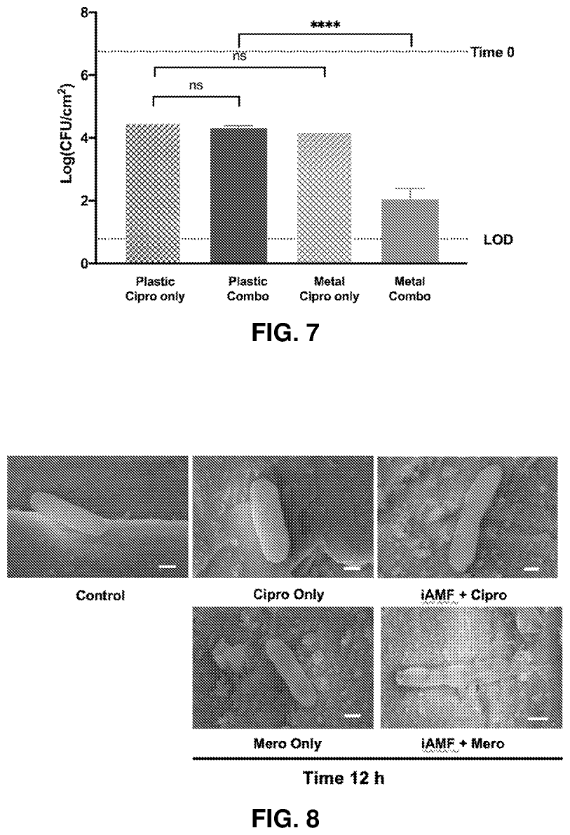

Having characterized the dynamics of ring heating with the iAMF system, Applicants investigated its ability to eradicate biofilm from the ring surface (FIGS. 2A-2D). As used herein, “eradicate” does not necessarily imply complete 100% removal or destruction of, for example, biofilm but can instead mean a significant reduction of, for example, biofilm. Each of the three iAMF treatments investigated (dotted blue lines) were able to reduce P. aeruginosa PA01 biofilm by approximately 1-2 log after each dose. However, between doses, CFU levels reverted to baseline. The rings exposed to 0.5 ug/ml of ciprofloxacin alone (solid black line) showed a steady CFU reduction over the first 12 hours of almost 3-log, followed by a plateauing after that. Strikingly, the iAMF exposures combined with ciprofloxacin (solid blue lines) demonstrated an unexpected result, namely a consistent reduction in biofilm down to the limit of detection. The reduction in CFU immediately after each dose was equal or larger for combined therapy compared with iAMF alone. In between the AMF doses at time 0 and 12 h, there was a further reduction in CFU, presumably as ciprofloxacin demonstrated enhanced activity in biofilm. Of note, the CFU reduction at 0 and 12 h were of a similar magnitude suggesting a consistent AMF treatment effect after each dose. This trend was observed for three different treatment strategies in which the target temperature (Tmax), and number of exposures (Nexp) was altered. Furthermore, more exposures were required at lower temperatures to observe an equivalent reduction in biofilm after 2 doses (FIGS. 2B, 2C, 2D). At 24 hours, the difference in CFU between the combined treatment group and all other groups was highly significant (p<0.0001). The same treatment strategy with iAMF at Tmax=65 degrees C. and ciprofloxacin combined was conducted on equally sized plastic rings or grade 5 titanium rings with P. aeruginosa biofilm. On plastic rings, biofilm CFU showed no significant difference when treated with iAMF and ciprofloxacin compared to ciprofloxacin incubation alone (FIG. 7). For biofilms on titanium rings, a material that is widely used in medical implants, biofilm reduction from iAMF and ciprofloxacin treatment was similar as that seen on stainless steel rings.

To evaluate whether a synergistic relationship exists between heat and antibiotics on biofilm, an experiment was conducted using a temperature-controlled water bath. Biofilms were exposed to varying durations of heating at specified temperatures, and then the CFU reduction in bacteria in the presence and absence of various antibiotic concentrations was quantified (see Supplementary Materials). The MBEC (minimal biofilm eradication concentration) was used to quantitatively study the synergistic effect of heat and ciprofloxacin as previously described (Reference 29). The results demonstrated synergy with fractional inhibitory concentration (FIC) index values that were below 0.5 (the definition for synergy) for various combinations of heat treatment time and ciprofloxacin concentrations at both 12 and 24 h post single heat treatment (References 30,31). This suggests that heat and ciprofloxacin display synergistic activity in the biofilm setting (FIG. 19) (Reference 29).

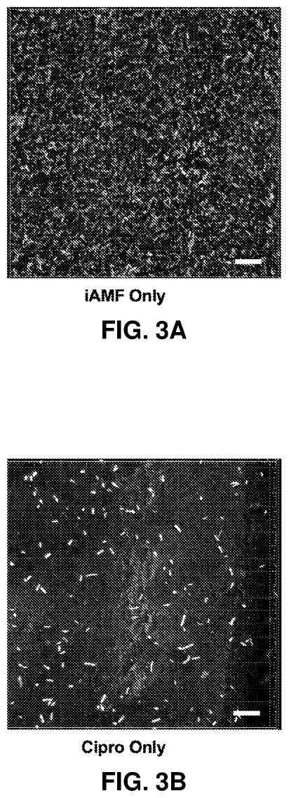

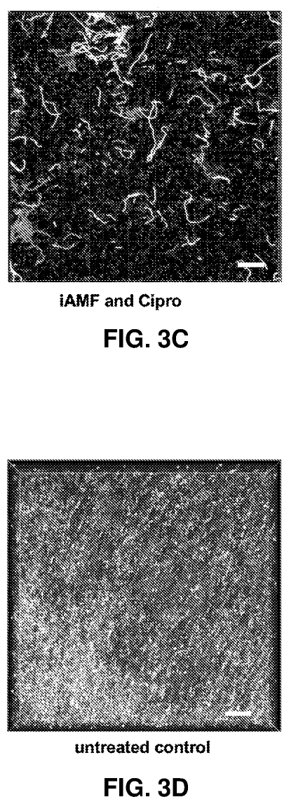

The enhanced reduction in biofilm to combined iAMF and antibiotics was also observed visually utilizing laser scanning confocal microscopy (FIGS. 3A-3D). GFP-PAO1 biofilms were treated using iAMF (Tmax=65 degrees C., Δtexp=5 minutes, Nexp=12) and 0.5 μg/mL ciprofloxacin GFP-PAO1 cells are represented in green and ConcanavalinA-Alexa Fluor 647 stained EPS was shown as red. This allowed for the morphology of bacterial cells to be observed under different treatment conditions. With ciprofloxacin only (FIG. 3B), the bacteria showed slight elongation compared to iAMF only (FIG. 3B) and control (FIG. 3D) at 12 h post-treatment. While the iAMF only group displayed diffuse ConcanavalinA-Alexa Fluor 647 stained EPS, the combined treatment of iAMF and ciprofloxacin (FIG. 3C) had less dense EPS staining. In addition, there were increased numbers of GFP-expressing cells that were elongated, a visual representation of Pseudomonas during quinolone treatment (References 32, 33).

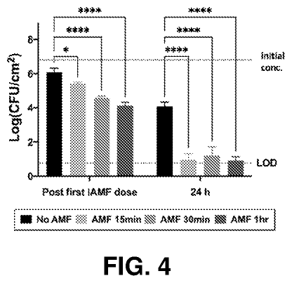

The impact of iAMF dose duration was investigated in more detail. P. aeruginosa biofilms were treated with iAMF (Tmax=65° C.) for dosing durations that ranged from 15 min to 1 h in combination with 0.5 μg/mL ciprofloxacin following the same treatment scheme as in FIG. 2A. Exposures were spaced apart by 5 minutes in each of the treatments. Immediately after combined iAMF and antibiotic treatment, reduction in CFU demonstrated a dose-dependent response with longer durations of iAMF resulting in greater decreases (FIG. 4, p=0.0318 for 15 min iAMF and p<0.0001 for 30 and 60 min iAMF). After 15 min of iAMF there was a 1.41 log reduction that increased to a 2.68 log reduction after the 1 h dose. After 24 h, there was 2.7 log reduction in biofilm treated with ciprofloxacin only, whereas the combination therapy achieved a greater than 5 log reduction, approaching the limit of detection for all iAMF treatment durations (p<0.0001 for all the three dosing durations). These results showed that biofilm can be effectively eliminated (i.e., significantly reduced) through combined treatment of iAMF and ciprofloxacin at a variety dosing durations. Indeed, only three iAMF exposures over 15 min together with ciprofloxacin were sufficient to effectively eliminate (i.e., significantly reduce) P. aeruginosa biofilm.

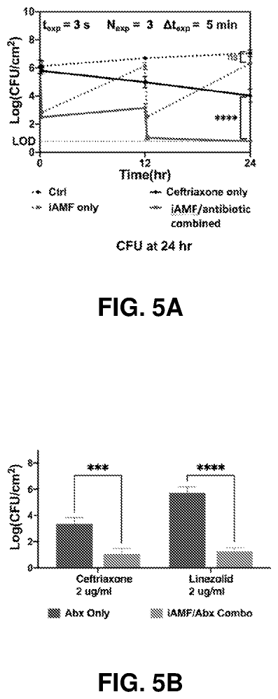

Similar patterns were observed for iAMF and antibiotic treatment of S. aureus biofilm. In addition to being a Gram-positive pathogen with several structural and metabolic differences compared to P. aeruginosa, S. aureus has clinical importance as one of the more common pathogens associated with metal implant infections. S. aureus (UAMS1) biofilms were treated with iAMF and antibiotics alone and in combination. Two antibiotics commonly used clinically were selected: ceftriaxone (2 μg/mL) and linezolid (2 μg/mL). These concentrations represented the minimum inhibitory concentration (MIC) for this strain. As in previous experiments, iAMF doses were delivered at 0 and 12 h. Each dose was composed of iAMF exposures with the following specifications: Tmax=65° C., Δtexp=5 min, tdose=15 min. For treatment with iAMF and 2 μg/mL ceftriaxone (FIG. 5A), biofilm CFU initially decreased by over 3 logs, suggesting that S. aureus biofilm has a greater sensitivity to iAMF dosing alone (3.29 log reduction) compared with P. aeruginosa (0.96 log reduction) with the same 15 min iAMF dose. As observed with PA01, in between doses, biofilm CFU returned to control levels for iAMF only groups. Incubation with ceftriaxone alone only led to approximately a 2-log reduction after 24 h. However, CFU reduction was significantly larger after 24 h when treated in combination with iAMF (p<0.0001) with CFU approaching the limit of detection. At 24 h, iAMF and ceftriaxone (2 μg/mL) or iAMF and linezolid (2 μg/mL) showed significantly lower CFU than with antibiotics alone (FIG. 5B; p=0.0004 for ceftriaxone and p<0.0001 for linezolid).

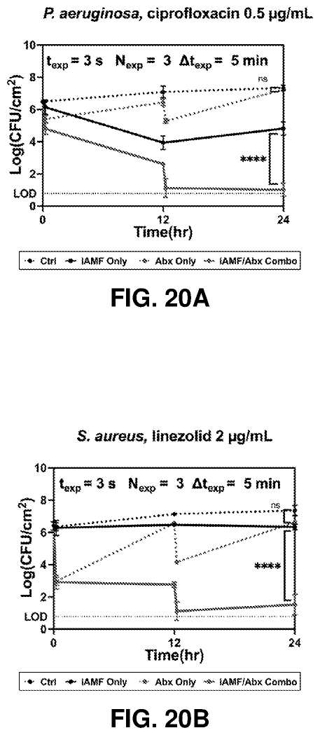

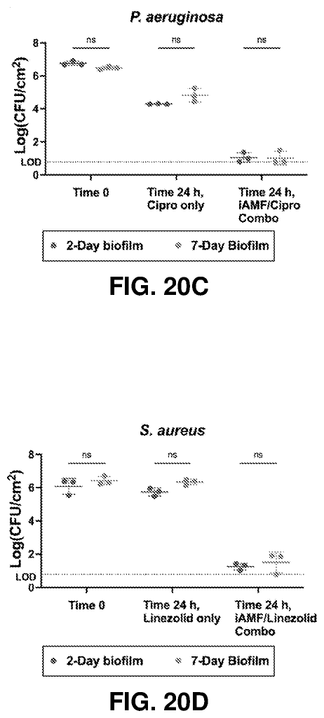

The age of the biofilm can vary in real-life clinical situations. Applicants investigated if the combination of iAMF and antibiotics could eliminate (i.e., significantly reduce) more mature biofilms beyond 48-h (2-day) old ones. 7-day P. aeruginosa (PAO1) and S. aureus (UAMS1) biofilms were cultured and the same experimental conditions were performed with iAMF at Tmax=65° C. as for 2-day biofilms. Similar reductions in CFU to 2-day biofilms were seen. When treated with the same iAMF dose (Tmax=65° C., Δtexp=5 min, tdose=15 min) as used with the 2-day biofilm and antibiotics (0.5 μg mL-1 ciprofloxacin for PAO1, and 2 μg mL-1 linezolid for UAMS1), the CFU change followed the same trend as was seen previously (FIG. 20A, 20B). There was no significant difference in the magnitude of the reduction of biofilm to iAMF and antibiotics for 2 and 7-day biofilms (FIG. 20C, 20D).

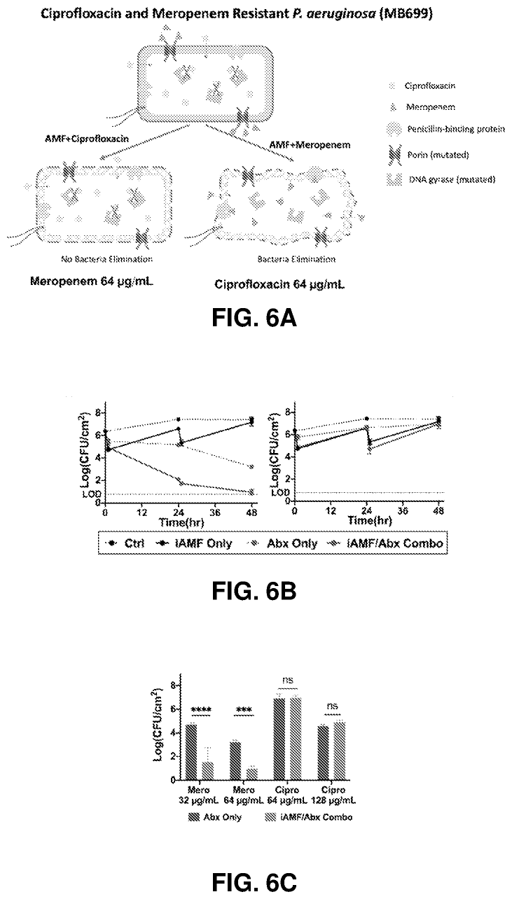

Antibiotic resistance is becoming increasingly common. Multidrug-resistant pathogens (MDR) only further complicate the treatment of biofilm-associated implant infections. The mechanism of the synergistic response between antibiotics and iAMF remain unknown. Applicants contended that one possible mechanism could relate to heat induced membrane disruption allowing for increased uptake of the antibiotic. To test whether iAMF could enhance antibiotic activity in MDR pathogens and enhance activity of specific antibiotics depending on the resistance mechanism, Applicants utilized an MDR P. aeruginosa isolate (MB699) that was genomically and phenotypically characterized. This clinical isolate was genome sequenced as described previously (Reference 34). It is an MDR isolate with a minimum inhibitory concentration (MIC) of 64 μg/mL for both ciprofloxacin and meropenem. Analysis of the genome revealed mutations in DNA gyrase (gyrA, p.Thr8311e) and topoisomerase IV (parC, p.Ser87Leu), which related to ciprofloxacin resistance, as well as in the porin (oprD, p.Thr103Ser, p.Lys115Thr, p.Phe170Leu, p.Glu185GIn, p.Pro186Glyfs*35, p.Thr187Profs*52, p.Va1189de1, p.Gly425Ala), which related to meropenem resistance. It was hypothesized that iAMF would enhance activity of meropenem but not ciprofloxacin. MB699 biofilm was treated with iAMF using the following parameters: Tmax=65 degrees C., Δtexp=5 min, Nexp=12, Ndose=2, Δtdose=24 h. Antibiotic administration followed the same protocol as for the PAO1 experiments and each antibiotic was dosed at its minimum inhibitory concentration. (FIG. 11). After 2 doses (0 and 24 h) and determining CFU at 48 h, bacterial burden approached the limit of detection for treatment with iAMF and meropenem, while ciprofloxacin and iAMF did not result in a further reduction of CFU compared to either iAMF or antibiotic alone (FIG. 6A). The reduction of meropenem with iAMF was also seen at sub-MIC concentrations (32 μg/mL) as well (p<0.0001; FIG. 6B). Increasing the concentrations of ciprofloxacin did not lead to enhanced CFU decreases in combination with iAMF. The effects of iAMF and meropenem versus ciprofloxacin on MB699 were observed by scanning electron microscopy (SEM). At 12 h post-treatment of MB699 biofilm with iAMF (Tmax=65 degrees C., Δtexp=5 min, Nexp=12) and continuous incubation with 64 μg/mL of ciprofloxacin or meropenem, biofilms were fixed as described and imaged. For treatment with ciprofloxacin, meropenem, or iAMF alone, no obvious morphological changes were observed in the bacteria. With iAMF and ciprofloxacin, some changes were observed, with slight lengthening of bacteria and increased wrinkling of the membrane. Treatment with iAMF and meropenem displayed fragmented and deformed bacterial cells (FIG. 8).

### Exhibit A Discussion

Although the effects of heat on bacterial killing have been known for years, major hurdles exist in order to utilize heat for antibacterial effects in the human body. Studies conducted by Applicants and others have demonstrated a strong therapeutic effect of heat generated via AMF and antibiotics on the eradication (i.e., significant reduction) of biofilm (References 20, 23, 24). A previous study by our group demonstrated that P. aeruginosa biofilm was more susceptible to ciprofloxacin after AMF treatment (Reference 22). Pijls et al. (References 24,35) reported similar results as was seen in this study, that there was an enhanced effect with AMF and antibiotics in Staphylococcus epidermidis and S. aureus biofilms on titanium alloy than with either treatment alone. One concern for the clinical adoption of AMF relates to therapeutic index, specifically the ability to reduce biofilm through thermal effects while minimizing neighboring tissue damage. Applicants developed a method, intermittent AMF, that could deliver AMF to infected metal implants that could aid in moving towards these goals of maintaining efficacy while limiting any toxicity. A premise of iAMF is that brief exposures to the surface of an implant with sufficient cool-down time in between exposures will result in a therapeutic dose capable of eradicating (i.e., significantly reducing) biofilm while protecting surrounding tissues from damage.

Applicants demonstrate that even iAMF exposures of a few seconds can reduce biofilm burden by 1-2 log. However, in the absence of more frequent dosing, there is regrowth back to baseline within 12 h. While more frequent dosing with iAMF could be used, an alternative approach utilized by embodiments includes using iAMF to enhance the activity of antibiotics. As has been previously reported, the antibiotics used in this study were not affected by the heat generated by iAMF and maintain stability at these temperatures (References 36, 37). In combination, iAMF and antibiotics resulted in a dramatic decrease in biofilm burden over either treatment alone. Importantly, this effect was not limited to one pathogen or one antibiotic. Applicants demonstrated that both clinically important Gram-positive (S. aureus) and Gram-negative pathogens (P. aeruginosa) and various antibiotics had their activity enhanced with iAMF. As diseases such as PJI are caused by a number of different bacterial pathogens, one goal of developing iAMF is to have a treatment that is efficacious regardless of the pathogen that is found. Applicants also demonstrated that the combination of iAMF and antibiotics can effectively eliminate (i.e., significantly reduce) biofilms of different ages. Importantly, this treatment effect was not seen on plastic rings, indicating the underlying principle of current generation between AMF and metals. In addition to quantitative reduction in bacterial burden, microscopy qualitatively supported the enhanced impact that iAMF and antibiotics had.

Biofilms are recalcitrant to antibiotic therapy for a number of reasons. This includes the difficulty in getting adequate concentrations of the drug to the target (bacteria) embedded within the biofilm matrix as well as difficulty in immune cells reaching these pathogens. This creates an environment where a biofilm-associated pathogen can be functionally antibiotic resistant. The increasing rate of antibiotic resistance that is being seen worldwide will only further complicate the treatment of biofilm-associated infections. One of the most striking findings of our studies was the ability to reduce certain multidrug-resistant bacteria based on the mechanism of resistance. Applicants utilized a genomically and phenotypically characterized Pseudomonas strain to begin to understand what the mechanism of action is that explains iAMF synergy with antibiotics. Applicants contended that iAMF disrupts bacterial membranes and that embodiments may reduce an MDR strain with an antibiotic if the mechanism of resistance was membrane-based (i.e., porins or efflux mechanisms). However, Applicants contended chromosomally based mechanisms of resistance (i.e., gyrase mutations) would not be impacted by an iAMF and antibiotic combination compared to either one alone. Embodiments support these contentions. Applicants were able to show a synergistic effect with iAMF and meropenem in this MDR strain with known mutations in the porin oprD but not with ciprofloxacin as the strain contained DNA gyrase mutations. Although, there are other potential mechanisms that could explain the interactions between iAMF and antibiotics in the biofilm setting, this data supports that membrane disruption is likely one important component.

Applicants effectively eliminated (i.e., significantly reduced) biofilm using iAMF with antibiotics on metal implants in vitro. The water bath experiments combined with defining heating exposure time as the “dose” of an antimicrobial did in fact support the position that synergistic interactions between iAMF and antibiotics are being seen.

Embodiments help address a number of somewhat previous unknowns regarding the ultimate deployment of iAMF in the clinical setting. This includes the optimal number of doses of iAMF that would lead to a durable treatment response as well as the optimal target temperature that would maintain efficacy while minimizing any potential safety concerns. Various embodiments described herein provide efficacious ranges for these values. Nevertheless, future and ongoing studies include exploring iAMF for safety and efficacy in a large animal model of implant infection. In addition, other possible mechanisms of this interaction remain to be explored.

### Exhibit A Materials and Methods

### In vitro AMF System

A custom-designed system composed of multiple solenoid coils was constructed to deliver programmed AMF exposures to stainless-steel rings with existing biofilm held in 50 mL conical tubes. The parameters of AMF exposure were assigned using custom-developed software operating on a personal computer. A function generator (33250A, Agilent Technologies) was used to produce an RF signal. The signal was input into a 1000 W RF amplifier (1140LA, Electronics & Innovation), and the amplified signal was directed to the appropriate coil using a USB-controlled relay system. Each coil was constructed using 0.25-inch diameter copper tubing formed into a 6-turn solenoid with 1 cm pitch between turns (FIG. 1A). The coil diameter was chosen to accommodate a 50 mL conical tube holding the infected ring and media. A plastic holder was included in each conical tube to hold the ring in place, so the orientation was maintained across all coils. The coils were driven electrically as a parallel resonant circuit using a capacitor selected to tune the resonant frequency to approximately 500 kHz. The working frequencies of the coils ranged from 507 to 522 kHz. A matching inductor was also included in series with the resonant circuit to transform the impedance of each coil to 50 ohms for efficient power transfer. The complete system included four insulated boxes each containing eight coils, enabling the treatment of up to 32 samples with iAMF in a single experiment. The coils worked at 8 Vpp with a 50% duty cycle (100 ms per 200 ms) for the experiments described herein. A circulating fan with integrated heater (Miller Manufacturing, MN, USA) was also incorporated into each box to keep the samples at 37° C. during extended length experiments.

Characterization and calibration. The strength of the alternating magnetic field in the coil was characterized using a commercial 2D magnetic field probe (AMF Lifesystems, Inc., MI, USA). A current probe (TRCP3000 Rogowski current probes, Tektronix Inc., OR, USA) was also used to measure the electrical current through the coils during operation.

To characterize AMF heating, uninfected metal rings were exposed for varying durations to reach desired maximum temperatures. The temperature of each ring exposed to AMF was measured using a fiber-optic temperature sensor (PRB-G40-2M-STM-MRI, Osensa Innovations, Burnaby, BC, Canada) attached to the center of the inner surface of the ring with high-temperature epoxy (Epotek 353ND, Epoxy Technologies, CA, USA). Tests were performed to confirm that the epoxy was unaffected by the AMF and did not produce false heating. Ring temperatures were recorded at a rate of 2 Hz using a laptop computer. The use of fiberoptic temperature sensors enabled accurate temperature characterization during AMF exposures since they are immune to electromagnetic interference.

Finite element analysis simulation Finite element simulations were performed using the commercial simulation software COMSOL Multiphysics (Comsol v5.5, Los Angeles, CA, USA) to model the interaction between AMF and a metal implant, and to study the uniformity and magnitude of AMF-induced heating. A quasi-static approximation of Maxwell's equation and Penne's bioheat transfer model was used for electromagnetic and thermal simulations. The thermal dose is calculated as cumulative equivalent minutes (CEM43) (Reference 38) which gives the time-temperature relation in equivalent minutes as

$$\\ C E M 43 = {{\int }^{{}^{t} final}}_{{t}_{0}} {R}^{{}^{} 43 - T ( t )} dt$$

where, R is the temperature dependence of the rate of cell death (R=0.5 for T>43, R=0.25 for 43≥T≥39), dt is the time interval, to and tfinal are initial and final heating periods respectively in minutes. The thermal toxicity due to implant heating is determined based on the tissue damage radius CEM 240 min (irreversible damage) (references 27,28) from the implant surface.

FIG. 1A shows the 3D physical model used for simulation of the metal ring in aqueous biological media in the coil. The coil geometry and current measured in the section above were used for 3D modeling and initial conditions of 37 degrees C. were selected for simulations. The physical properties used for simulations are listed in Table S1 25,26. Simulations were performed using free tetrahedral meshing with boundary layers. Grid independent studies were performed from coarser to finer meshes, settling on an optimal number of 186,634 elements to be used for analysis.

### In vitro AMF Treatment

iAMF treatment parameters. The structure and timing of iAMF treatments is shown in FIG. 1B. Treatments were organized as a series of doses (Ndose), each separated by a fixed time (Δtdose). The length of an iAMF dose ranges from 15 min to a few hours. Ndose is the number of doses in the whole treatment. Each dose was composed of multiple AMF exposures. During each exposure, AMF is on for a few seconds and the rings are heated. The exposures are separated by fixed time intervals (Δtexp) to allow rings to cool to the initial temperature between exposures. (Nexp) is the number of exposures performed in one iAMF dose. The heating from a typical exposure is shown with a specified target temperature, Tmax, and a cooldown back to the baseline temperature over 3-5 minutes. The temperature profile for three different Tmax values (50, 65, and 80) are also shown. The target temperatures were achieved by varying the duration of AMF exposure in the coil. For iAMF treatments at Tmax=80° C., the temperature reached 80° C. in 6 s and was held until 12 s during the initial construction of the system. Therefore, this iAMF heating pattern was used in the Tmax=80° C. iAMF experiments described below.

Biofilm was grown on stainless steel rings (316 L, ¾″ OD, 0.035″ wall thickness, 0.2″ height, cut from McMaster Carr, P/N 89785K857, USA) or Titanium rings (Grade 5, ¾″ OD, 0.035″ wall thickness, 0.2″ height, cut from McMaster Carr, P/N 89835K93, USA) using the Gram-negative pathogen P. aeruginosa (PAO1: ATCC strain. PAO1-GFP: provided by Joanna Goldberg, MB699: provided by Sam Shelburne) or Gram-positive pathogen S. aureus (UAMS1, provided by M. Smeltzer). For P. aeruginosa biofilm, an isolated colony was inoculated into 3 mL of cation-adjusted Mueller Hinton II (MHII) media (Becton-Dickinson by Thermo-Fisher Scientific) and incubated at 37° C. for 18 h at 220 RPM. A working solution was made by adding culture to sterile phosphate-buffered saline (PBS). The bacterial concentration was adjusted with MHII using a UV spectrophotometer (Genesys 20, Thermal Scientific) at 600 nm until the optical density (OD) read between 0.07 and 0.08, indicating a concentration of ˜108 CFU mL-1. The working solution was then diluted to obtain a bacterial concentration of 5×105 CFU mL-1. Biofilm was prepared on each metal ring by placing the ring in 5 mL of the bacterial solution in a 50 mL conical tube. The submerged ring was then incubated at 37° C. for 48 h at 110 RPM in a shaking incubator (Innova42, New Brunswick Scientific). Media was replenished midway at 24 h by exchanging the solution with 5 mL of fresh MHII. Biofilm prepared with S. aureus followed the same protocol using Tryptic Soy Broth (TSB, Becton-Dickinson by Thermo-Fisher Scientific). Biofilms other than the 7-day old biofilm in this study were prepared using this protocol. For the 7-day old biofilm, the rings were cultured similarly but the culture time was prolonged to 7 days with media replenishment every 24 h.

Biofilm preparation, treatment and quantification. The multi-coil system described above was used to investigate the response of biofilm (P. aeruginosa or S. aureus) grown on stainless-steel rings to AMF. Biofilm-coated rings were transferred to 50 mL conical tubes each with 10 mL fresh media containing antibiotics at set concentrations. Prior to transfer, the tubes of fresh media were pre-warmed in the multi-coil system to 37° C. After the rings were transferred to the tubes, sterile 3D-printed ring holders were placed on the top of the rings to maintain their orientation in the coil during AMF exposures. The rings were then exposed to intermittent AMF according to treatment protocols. After each intermittent dose, the rings were rinsed in 10 mL fresh antibiotic-containing media to remove planktonic bacteria. Then the rings were transferred again to 10 mL of fresh antibiotic-containing media and incubated at 37 degrees C. After a fixed time period (typically 12-24 h), the rings were exposed to a second dose of AMF using the same protocol, and the rings were again incubated in 10 mL media with antibiotics at 37 degrees C. for another 12-24 h. Before and after each iAMF dose, and at the treatment endpoint, the rings were harvested and rinsed in 5 mL PBS and then transferred to 4 mL PBS. The rings were sonicated in an ultrasonic water bath for 5 min and bacterial density on the ring surface was quantified by plating on blood agar plates (TSA w/sheep blood, Thermo Fisher Scientific) using a standard serial dilution drip method. Three biological replicates were obtained for each experimental condition, and three technical replicates were utilized per experiment. Control groups for all studies included rings unexposed to antibiotics or AMF, and rings exposed to iAMF or antibiotics as monotherapy. All control groups went through the multiple rinse and transfer steps to account for any bacterial loss. A two-way ANOVA model was used to compare bacterial burden at different time points for single or combined therapy.

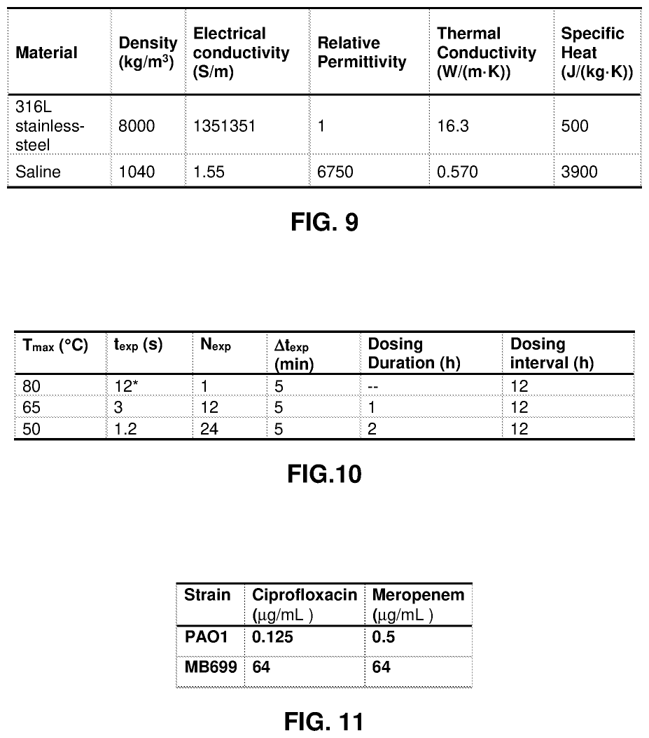

A final control group involved iAMF treatment of infected plastic rings with the same dimensions as the metal rings, to establish the observed effects were arising from the interactions between AMF and metal. See FIGS. 7-11 for further details.

Experiments with different AMF target temperatures (Tmax) Three unique iAMF treatment algorithms were delivered to rings infected with PA01 biofilm. The rings were incubated with ciprofloxacin (0.5 μg/mL) in 10 mL MHII media at 37 degrees C. for all treatments. Each treatment reached a different target temperature and had a different number of exposures in each dose, as described in FIG. 10. Doses were repeated at 0 and 12 hours.

Although multiple parameters were varied in each setting, the goal was to balance the maximum temperature with the number of exposures to maintain a level of safety. These choices were governed by ongoing bioheat transfer simulations in our group (not shown). Each of these AMF treatment combinations were predicted to be safe in terms of tissue damage around the implant base on simulation.

Experiments with variable AMF dose durations in combination with antibiotic treatment Biofilms of P. aeruginosa strain PAO1 were prepared on stainless steel rings using the same culturing protocol as above and incubated with 0.5 μg/mL of ciprofloxacin in 10 mL MHII media at 37 degrees C. Rings were exposed to iAMF to a Tmax of 65 degrees C. with an exposure interval of 5 min. The duration of each iAMF dose ranged from 15 min to 1 h (3 to 12 exposures). Doses were delivered at 0 and 12 hours and ring biofilm burden was quantified at various time points as above. For S. aureus experiments, biofilm of UAMS1 were prepared on stainless steel rings according to the culturing protocol and incubated with 2 μg/mL of ceftriaxone or 2 μg/mL of linezolid, in 10 mL TSB media. The rings were exposed to iAMF to a Tmax of 65 degrees C. with 5 min between each exposure, for a duration of 15 min per dose (3 exposures). Doses were delivered at 0 and 12 hours and biofilm burden was quantified at 24 hours.

Treatment of resistant strains with combined iAMF and antibiotics Biofilms of MB699, an MDR-strain of P. aeruginosa, were incubated with ciprofloxacin (64 or 128 μg/ml) or meropenem (32 or 64 μg/ml of) in 10 mL MHII media. The rings were exposed to iAMF to a Tmax of 65 degrees C. with 5 min between exposures for a duration of 1 h per dose. Doses were delivered at 0 and 24 hours and ring biofilm burden was quantified at 48 hours.

### Imaging

Laser Scanning Confocal Microscopy Biofilms cultured from green-fluorescent protein (GFP) expressing PAO1 P. aeruginosa (GFP-PAO1) were prepared on rings using the above protocol, then exposed to iAMF (Tmax=65 degrees C., Δtexp=5 min, dosing duration 1 h) and incubated in 10 mL MHII media with 0.5 μg/mL ciprofloxacin for 12 h. After rinsing in 5 mL DPBS, rings were then fixed in 5% glutaraldehyde (Sigma Aldrich, St. Louis, MO) at 37° C. for 30 min and protected from light. Rings were then rinsed in 5 ml of DPBS to remove excess glutaraldehyde and incubated in 200 μg/mL ConcanavalinA-Alexa Fluor 647 conjugate (Life Technologies, Grand Island, NY) for 15 min at room temperature at dark to stain the EPS. After staining, rings were mounted on a 50 mm glass bottom plate and images were captured with a Zeiss LSM880 Airyscan laser confocal microscope. The GFP-PA01 bacteria and ConA-stained EPS were imaged using a 40× objective lens. Multiple regions of the ring surface were randomly selected, and Z-stacks were acquired with slice step size of 0.5 μm. Before image processing, the z-stacks were deconvolved using Autoquant×3 (Media Cybernetics, MD, USA) to improve the image resolution in X, Y and Z directions. The deconvolved images were analyzed with Imaris x64 9.1.2 (Bitplane AG, Zurich, Switzerland).

Scanning Electron Microscopy (SEM) Biofilms cultured from P. aeruginosa (MB699) were prepared on rings and exposed to iAMF (Tmax=65 degrees C., Δtexp=5 min, dosing duration 1 h) and incubated in 10 mL MI-III media with 64 μg/mL ciprofloxacin or 64 μg/mL meropenem for 12 h. Then the rings with biofilm were prepared for SEM, following a similar protocol described previously (Reference 40). The rings were carefully transferred to 4 mL PBS, rinsed in 4 mL of 0.1 M sodium cacodylate buffer three times and fixed for 24 h in 4 mL of 2% glutaraldehyde, 2% paraformaldehyde in 0.1 M sodium cacodylate buffer. After rinsing in 4 mL of cacodylate buffer three times, the samples were re-fixed in 4 mL of 2% osmium in 0.1 M sodium cacodylate buffer for 2 h. Then the rings were further rinsed with 4 mL of deionized water five times and dehydrated at room temperature in five steps by placing the rings in 4 mL of 50, 70 (twice), 85, 95 (twice) and 100% ethanol respectively for 5 min per solution. The rings were then transferred to 4 mL of 25, 50, 75 and 100% (twice) hexamethyldisilazane (HMDS) in ethanol consecutively for 15 min each. Finally, the samples were left to dry for 24 h in a fume hood. The specimens were mounted on aluminum stubs, gold/palladium sputter coated, and examined using a Zeiss Sigma VP scanning electron microscope. The images were acquired at 10 kV with magnification of approximately 35000×.

Statistics. Significance was determined as described for in vitro AMF treatment by two-way ANOVA followed by Tukey's multiple comparisons test. The “n” indicates the number of biological replicates. 2 or 3 technical replicates were conducted for each biological replicate. All analyses were performed using GraphPad Prism version 8.4.3 (San Diego, CA), and a p-value of<0.05 was considered statistically significant.

### Exhibit A Supplementary Materials

Determining epoxy immunity (Epotek 353ND) to iAMF A fiberoptic thermal sensor was glued with the Epotek 353ND epoxy at the tip and placed in 10 mL of DPBS. A bare sensor was placed in the DPBS as well. The distance between the tips of the two sensors was 1 cm. iAMF (Tmax=65° C.) was applied for 10 min and the temperature reading from the two sensors was recorded and compared.

Determination of synergy between heat and antibiotics in biofilm. The synergy of heat and ciprofloxacin in biofilm was determined using the fractional inhibitory concentration (FIC) index (Supplemental References 1-3). The FIC index was calculated based on the minimal biofilm eradication concentration (MBEC), defined as the lowest concentration of an antimicrobial substance that eradicates 99.9% of biofilm-embedded bacteria (3-log reduction in CFU mL-1) compared to growth controls. The thermal treatment time was treated as the antimicrobial substance dose, and the MBEC for heat treatment was defined as the shortest treatment time that eliminated 99.9% of biofilm-embedded bacteria (Supplemental Reference 4). Thus, the equation for the FIC index calculation with heat treatment and antibiotics can be derived: FIC=(CHeat/MBECHeat)+(CAbx/MBECAbx), where MBECHeat and MBECAbx are the MBECs of heat treatment and antibiotics concentration alone, respectively, and CHeat and CAbx are thermal treatment time and antibiotics concentration in combination, respectively. FIC values of £0.5 were considered to be a synergistic effect, values of >0.5 and <4 indicated no interaction or additivity, and values of greater than or equal to 4 indicated an antagonistic effect (Supplemental References 3,4).

A temperature-controlled water bath (Model 1235, VWR Scientific) was used to conduct the heat treatment. 50 mL tubes with 10 mL fresh MI-III were placed in the water bath and prewarmed to 65° C. containing ciprofloxacin at certain concentrations. PAO1 biofilms were prepared as described before. PAO1 biofilm-coated rings were transferred to pre-warmed 50 mL conical tubes and exposed in heated media for the targeted duration of time. After the heat exposure, the rings with biofilm were immediately transferred to 10 mL fresh media with ciprofloxacin in 50 mL conical tubes at set concentrations at 37° C. Then the rings were incubated at 37° C. After 12 h or 24 h, the rings were harvested and rinsed in 5 mL sterile PBS and then transferred to 4 mL PBS. After sonicating for 5 min in an ultrasonic bath, the bacterial density on the ring was enumerated using standard serial plating methods to determine the CFU cm-2.

### Further Discussion of Embodiments

AMF is a non-invasive approach to treat implant associated infections, in which an external transducer coil generates time-varying AMF in the vicinity of a metal implant in the body. The AMF generates surface electrical currents on the implant, which may eradicate (i.e., significantly reduce) pathogens. In the case of an infected implant, bacteria, which may be in the form of a biofilm, adheres to the surface. This localized current can be used to eradicate (i.e., significantly reduce) pathogens or sensitize them to antimicrobial treatment.

An embodiment involves the induction of very high currents for very short periods of time, resulting in little to no heating of surrounding tissue, but with similar antibacterial effect as previous treatment methods which result in higher tissue temperatures. Thus, embodiments treat a problem (tissue damage due to heat when trying to treat biofilms) using lower temperature and antibacterial effects of AMF to reduce the risk of thermal damage to surrounding tissues.

Embodiments show that the duty cycle of AMF exposure has an impact on temperature elevation. When exposing a metal ring to AMF, an exposure of 1 ms duration with a period of 1 sec (0.1% duty cycle) resulted in less than 5-6 degrees C. total temperature elevation over 2 hours. Similar heating was observed for 0.1% duty cycle exposures with different durations (10 ms ever 10 sec, 40 ms every 40 s). Embodiments demonstrate a CFU reduction for the 40 ms exposure in the presence of antibiotics.

Pulsed exposures have increased effect when applied in conjunction with antibiotics.

The use of brief pulsed exposures can generate high currents on an implant without a significant temperature elevation. This enhances the safety of embodiments compared to using exposures designed to reach therapeutic temperatures (60-80 degrees C.). However, with some embodiments longer exposures are required with brief exposures to achieve a therapeutic effect. Further, the effect of some embodiments is dependent on the concentration of antibiotics administered. A hybrid approach (which uses a temperature sufficient to generate inflammatory responses, which in turn trigger the immune system) is used in some embodiments. The temperature elevation can be controlled by changing the duty cycle of the treatment.

While previous disclosures discuss the utilization of heat as directly antimicrobial, the mechanism of action of low temperature embodiments may include: (a) mechanical disruption of the biofilm matrix (which allows for better penetration of antibiotics and the ability of the antibiotic to reach its target), (b) stimulation of otherwise ‘dormant’ metabolically inactive organisms that now become sensitive to a particular antimicrobial, (c) or a combination of the above.

Just as is seen with high temperature AMF, low-temperature AMF may be synergistic with multiple antimicrobials and may not be restricted to a single chemical class of drugs. Therefore, embodiments may be broadly applicable for bacterial and fungal infections or any pathogen that can form a biofilm on a metallic implant.

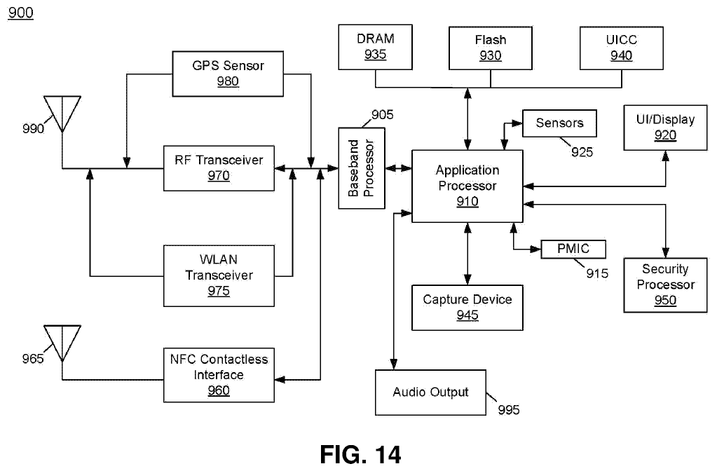

FIG. 14 includes a block diagram of an example system with which embodiments can be used. As seen, system 900 may be a smartphone or other wireless communicator or any other Internet of Things (loT) device. A baseband processor 905 is configured to perform various signal processing with regard to communication signals to be transmitted from or received by the system. In turn, baseband processor 905 is coupled to an application processor 910, which may be a main CPU of the system to execute an OS and other system software, in addition to user applications such as many well-known social media and multimedia apps. Application processor 910 may further be configured to perform a variety of other computing operations for the device.

In turn, application processor 910 can couple to a user interface/display 920 (e.g., touch screen display). In addition, application processor 910 may couple to a memory system including a non-volatile memory, namely a flash memory 930 and a system memory, namely a DRAM 935. As further seen, application processor 910 also couples to a capture device 945 such as one or more image capture devices that can record video and/or still images.

A universal integrated circuit card (UICC) 940 comprises a subscriber identity module, which in some embodiments includes a secure storage to store secure user information. System 900 may further include a security processor 950 (e.g., Trusted Platform Module (TPM)) that may couple to application processor 910. A plurality of sensors 925, including one or more multi-axis accelerometers may couple to application processor 910 to enable input of a variety of sensed information such as motion and other environmental information. In addition, one or more authentication devices may be used to receive, for example, user biometric input for use in authentication operations.

As further illustrated, a near field communication (NFC) contactless interface 960 is provided that communicates in an NFC near field via an NFC antenna 965. While separate antennae are shown, understand that in some implementations one antenna or a different set of antennae may be provided to enable various wireless functionalities.

A power management integrated circuit (PMIC) 915 couples to application processor 910 to perform platform level power management. To this end, PMIC 915 may issue power management requests to application processor 910 to enter certain low power states as desired. Furthermore, based on platform constraints, PMIC 915 may also control the power level of other components of system 900.

To enable communications to be transmitted and received such as in one or more internet of things (IoT) networks, various circuits may be coupled between baseband processor 905 and antenna 990. Specifically, a radio frequency (RF) transceiver 970 and a wireless local area network (WLAN) transceiver 975 may be present. In general, RF transceiver 970 may be used to receive and transmit wireless data and calls according to a given wireless communication protocol such as 5G wireless communication protocol such as in accordance with a code division multiple access (CDMA), global system for mobile communication (GSM), long term evolution (LTE) or other protocol. In addition, a GPS sensor 980 may be present, with location information being provided to security processor 950. Other wireless communications such as receipt or transmission of radio signals (e.g., AM/FM) and other signals may also be provided. In addition, via WLAN transceiver 975, local wireless communications, such as according to a Bluetooth™ or IEEE 802.11 standard can also be realized.

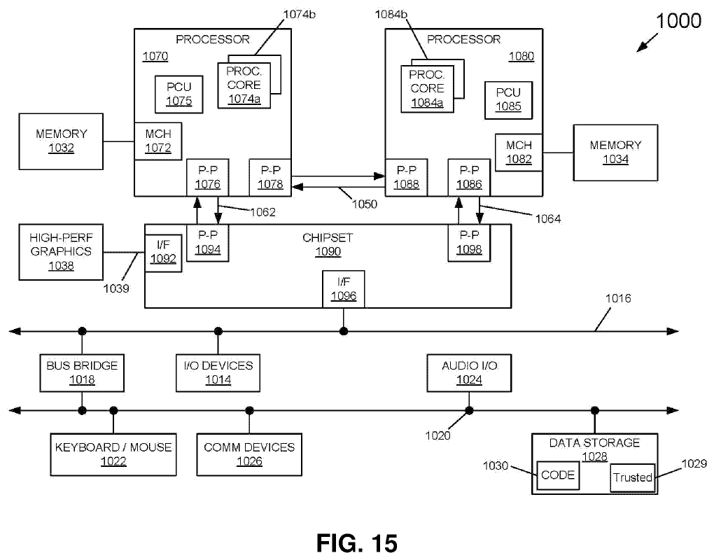

FIG. 15 shows a block diagram of a system in accordance with another embodiment of the present invention. Multiprocessor system 1000 is a point-to-point interconnect system such as a server system, and includes a first processor 1070 and a second processor 1080 coupled via a point-to-point interconnect 1050. Each of processors 1070 and 1080 may be multicore processors such as SoCs, including first and second processor cores (i.e., processor cores 1074a and 1074b and processor cores 1084a and 1084b), although potentially many more cores may be present in the processors. In addition, processors 1070 and 1080 each may include power controller unit 1075 and 1085. In addition, processors 1070 and 1080 each may include a secure engine to perform security operations such as attestations, loT network onboarding or so forth.

First processor 1070 further includes a memory controller hub (MCH) 1072 and point-to-point (P-P) interfaces 1076 and 1078. Similarly, second processor 1080 includes a MCH 1082 and P-P interfaces 1086 and 1088. MCH's 1072 and 1082 couple the processors to respective memories, namely a memory 1032 and a memory 1034, which may be portions of main memory (e.g., a DRAM) locally attached to the respective processors. First processor 1070 and second processor 1080 may be coupled to a chipset 1090 via P-P interconnects 1062 and 1064, respectively. Chipset 1090 includes P-P interfaces 1094 and 1098.

Furthermore, chipset 1090 includes an interface 1092 to couple chipset 1090 with a high-performance graphics engine 1038, by a P-P interconnect 1039. In turn, chipset 1090 may be coupled to a first bus 1016 via an interface 1096. Various input/output (I/O) devices 1014 may be coupled to first bus 1016, along with a bus bridge 1018 which couples first bus 1016 to a second bus 1020. Various devices may be coupled to second bus 1020 including, for example, a keyboard/mouse 1022, communication devices 1026 and a data storage unit 1028 such as a non-volatile storage or other mass storage device. As seen, data storage unit 1028 may include code 1030, in one embodiment. As further seen, data storage unit 1028 also includes a trusted storage 1029 to store sensitive information to be protected. Further, an audio I/O 1024 may be coupled to second bus 1020.

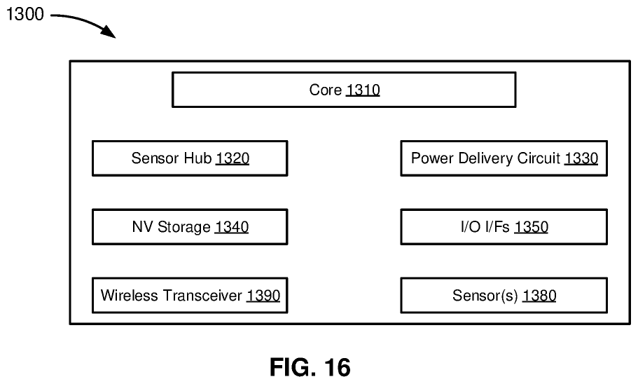

FIG. 16 depicts an loT environment that may include wearable devices or other small form factor loT devices. In one particular implementation, wearable module 1300 may be an Intel® Curie™ module that includes multiple components adapted within a single small module that can be implemented as all or part of a wearable device. As seen, module 1300 includes a core 1310 (of course in other embodiments more than one core may be present). Such a core may be a relatively low complexity in-order core, such as based on an Intel Architecture® Quark™ design. In some embodiments, core 1310 may implement a Trusted Execution Environment (TEE). Core 1310 couples to various components including a sensor hub 1320, which may be configured to interact with a plurality of sensors 1380, such as one or more biometric, motion, environmental or other sensors. A power delivery circuit 1330 is present, along with a non-volatile storage 1340. In an embodiment, this circuit may include a rechargeable battery and a recharging circuit, which may in one embodiment receive charging power wirelessly. One or more input/output (IO) interfaces 1350, such as one or more interfaces compatible with one or more of USB/SPI/12C/GPIO protocols, may be present. In addition, a wireless transceiver 1390, which may be a Bluetooth™ low energy or other short-range wireless transceiver is present to enable wireless communications as described herein. In different implementations a wearable module can take many other forms. Wearable and/or loT devices have, in comparison with a typical general-purpose CPU or a GPU, a small form factor, low power requirements, limited instruction sets, relatively slow computation throughput, or any of the above.

Embodiments may be used in many different types of systems. For example, in one embodiment a communication device can be arranged to perform the various methods and techniques described herein. Of course, the scope of the present invention is not limited to a communication device, and instead other embodiments can be directed to other types of apparatus for processing instructions, or one or more machine readable media including instructions that in response to being executed on a computing device, cause the device to carry out one or more of the methods and techniques described herein.

Program instructions may be used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as (a) a computer program product that may include one or more machine readable media having stored thereon instructions that may be used to program a processing system or other electronic device to perform the methods or (b) at least one storage medium having instructions stored thereon for causing a system to perform the methods. The term “machine readable medium” or “storage medium” used herein shall include any medium that is capable of storing or encoding a sequence of instructions (transitory media, including signals, or non-transitory media) for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving or signal preserving storage. A medium may include any mechanism for storing, transmitting, or receiving information in a form readable by a machine, and the medium may include a medium through which the program code may pass, such as antennas, optical fibers, communications interfaces, and the like. Program code may be transmitted in the form of packets, serial data, parallel data, and the like, and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

A module as used herein refers to any hardware, software, firmware, or a combination thereof. Often module boundaries that are illustrated as separate commonly vary and potentially overlap. For example, a first and a second module may share hardware, software, firmware, or a combination thereof, while potentially retaining some independent hardware, software, or firmware. In one embodiment, use of the term logic includes hardware, such as transistors, registers, or other hardware, such as programmable logic devices. However, in another embodiment, logic also includes software or code integrated with hardware, such as firmware or micro-code.

Various examples of embodiments are now addressed.

Example 1. A system comprising: at least one alternating magnetic field (AMF) transmitter configured to apply one or more AMF pulses to a metallic implant; at least one function generator; at least one processor; and at least one machine-readable medium having stored thereon data which, if used by the at least one processor, causes the at least one processor, the at least one function generator, and the at least one transmitter to perform operations comprising communicating a plurality of AMF pulses to the metallic implant; wherein each of the plurality of AMF pulses has a duty cycle of less than 1% and a period of between 1 ms and 60 seconds.

A “duty cycle” or power cycle is the fraction of one “period” in which a signal or system is active. Duty cycle is commonly expressed as a percentage or a ratio. A period is the time it takes for a signal to complete an on-and-off cycle. A duty cycle (ratio) may be expressed as: D=(PW)/T, where D is the duty cycle, PW is the pulse width (pulse active time), and T is the total period of the signal. Thus, a 60% duty cycle means the signal is on 60% of the time but off 40% of the time. The “on time” for a 60% duty cycle could be a fraction of a second, a day, or even a week, depending on the length of the period.

In other embodiments, each of the plurality of AMF pulses has a duty cycle of less than 1, 2, 3, 4, 5, or 6%. In other embodiments, each of the plurality of AMF pulses has a period of between 0.5 msecs to 20 seconds.

Another version of Example 1. A system comprising: at least one alternating magnetic field (AMF) transmitter configured to apply one or more AMF pulses to a metallic implant; at least one function generator; at least one processor; and at least one machine-readable medium having stored thereon data which, if used by the at least one processor, causes the at least one processor, the at least one function generator, and the at least one transmitter to perform operations comprising communicating a plurality of AMF pulses to the metallic implant; wherein each of the plurality of AMF pulses has a duty cycle of less than 1% and a period of between 200 ms and 60 seconds.

Example 2. The system of example 1, wherein the plurality of AMF pulses has a magnetic field no greater than 5 milliTesla (mT).

Example 3. The system according to any of examples 1-2, wherein each of the plurality of pulses has a pulse width of between 2 ms and 50 ms.

Example 4. The system according to any of examples 1-3, wherein the operations comprise communicating the plurality of AMF pulses to the metallic implant for a duration of at least 30 minutes.

Example 5. The system according to any of examples 1-4, wherein: the at least one machine-readable medium comprises a first protocol configured for a first metallic implant and a second protocol configured for a second metallic implant; the first metallic implant has a first physical contour and the second metallic implant has a second physical contour that is unequal to the first physical contour; the first protocol includes a first duty cycle and the second protocol includes a second duty cycle that is unequal to the first duty cycle.

Another version of Example 5. The system of example 4, wherein: the at least one machine-readable medium comprises a first protocol configured for a first metallic implant and a second protocol configured for a second metallic implant; the first metallic implant has a first magnitude of a physical characteristic and the second metallic implant has a second magnitude of the physical characteristic that is unequal to the first magnitude of the physical characteristic; the first protocol includes a first duty cycle and the second protocol includes a second duty cycle that is unequal to the first duty cycle.

Another version of Example 5. The system of example 4, wherein: the at least one machine-readable medium comprises a first protocol configured for a first metallic implant and a second protocol configured for a second metallic implant; the first metallic implant has a first magnitude of a physical characteristic and the second metallic implant has a second magnitude of the physical characteristic that is unequal to the first magnitude of the physical characteristic; the first protocol includes a first magnitude of a therapeutic characteristic and the second protocol includes a second magnitude of the therapeutic characteristic that is unequal to the first magnitude of the therapeutic characteristic.

For example, software may provide the user via a user interface, to use different treatment protocols for different devices. Two different protocols may be used for two different sizes of the same knee implant. Two different protocols may be used for two different brands of the same knee implant (one device from manufacture1 and another from manufacturer2).

Example 5.1 The system of example 5, wherein the physical characteristic includes one of density (kg/m\hat{ }3), electrical conductivity (S/m), relative permittivity, or thermal conductivity (W/(m·K)), specific heat (J/(kg·K)).

Example 5.2 The system according to any of examples 1-5.1, wherein the therapeutic characteristic includes one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

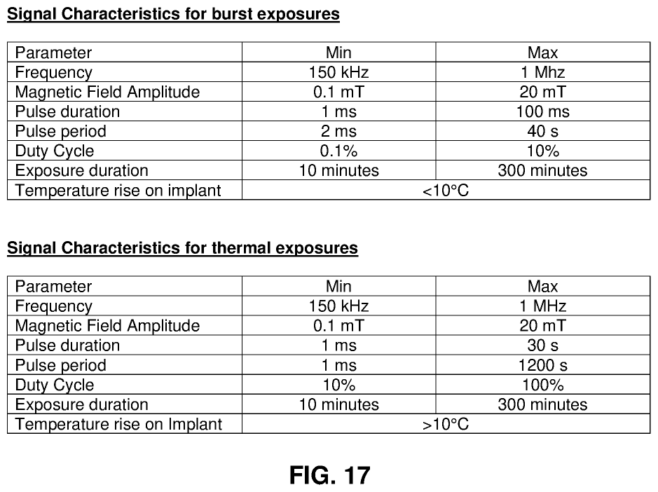

Embodiments are manyfold and include various ranges and combinations of ranges such as those found in the following table. In other words, different frequencies within the ranges in the table below may be combined various exposure durations (or other parameters) within the ranges in the table of FIG. 17.

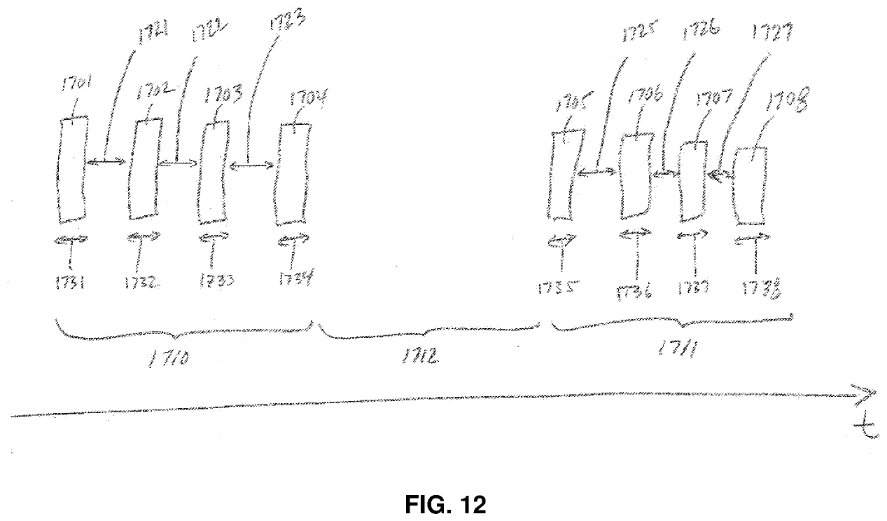

In FIG. 12, N_{dose}=2 (including dose 1710 and dose 1711). Δt_{dose} is indicated at 1712. texp is indicated at 1731, 1732, 1733, 1734, 1735, 1736, 1737, 1738. In an embodiment these values are equal to one another but in other embodiments these values are not all equal to one another. Nₑₓₚ=4 and includes exposures 1701, 1702, 1703, 1704 for dose 1710 and exposures 1705, 1706, 1707, 1708, for dose 1711. Nexp is the same for each of doses 1710, 1711 but may vary between the doses in other embodiments. Δtₑₓₚ is indicated at 1721, 1722, 1723, 1725, 1726, 1727. In an embodiment these values are equal to one another but in other embodiments these values are not all equal to one another.

In FIG. 12 an example period includes tₑₓₚ+Δtₑₓₚ. A duty cycle may be expressed as: D=(PW)/T, which in this case includes D=(tₑₓₚ)/(tₑₓₚ+Δtₑₓₚ).

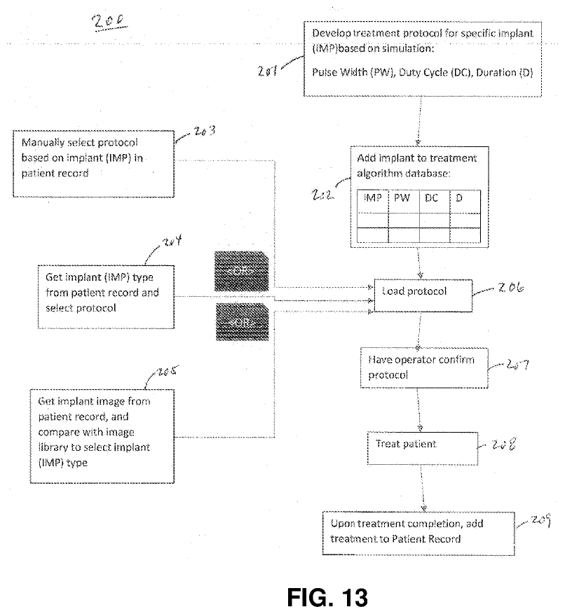

FIG. 13 addresses a method 200 that can be executed by at least one processor. As addressed above, various protocols may be determined. For example, a first protocol including a certain duty cycle or other therapeutic characteristic may be designed for a first manufacturer's metallic stent and a second protocol including a certain duty cycle or other therapeutic characteristic may be designed for a second manufacturer's metallic stent. For example, a first protocol including a certain duty cycle or other therapeutic characteristic may be designed for use with a first antibiotic and a second protocol including a certain duty cycle or other therapeutic characteristic may be designed for use with a second antibiotic. For example, a first protocol including a certain duty cycle or other therapeutic characteristic may be designed for use with a first dosage amount of a first antibiotic and a second protocol including a certain duty cycle or other therapeutic characteristic may be designed for use with a second dosage amount of the first antibiotic. For example, a first protocol including a certain duty cycle or other therapeutic characteristic may be designed for use with a first material (e.g., silver nanoparticles) coating the implant and a second protocol including a certain duty cycle or other therapeutic characteristic may be designed for use with a second material coating the implant. These protocols may be based on simulations, like those addressed in Exhibit A. As shown in block 201, protocols may vary by, in the least, pulse width, duty cycle, duration of dose, and the like.

In block 202 various protocols may be stored in a database, such as the memories addressed in FIG. 14, 15, or 16.

In block 203 a user may select a protocol based on his or her knowledge of the implant to be treated. The protocol may also be selected based on other patient specific details, such as a patient's age or weight, type of biofilm (e.g., what type of bacteria are causing biofilm), where in the patient the implant is located, and the like. However, in block 204 this information may be imported from, for example, a medical record such that importing the medical implant information leads to automatic selection of a protocol corresponding to that implant. In block 205 imaging may be used to identify the implant and upon identification, a protocol may be suggested that is specific to that implant. This image identification may be compared to information stored in a medical record. Based on the comparison, a user may select the proper protocol (which may be listed in a list that is a subset of all the protocols). Protocols may propose acceptable ranges within which a user may select a parameter (e.g., a max Temp between 60 and 70 degrees C. where the user selects 68 degrees C.).

A protocol is then loaded in block 206, along with protocol confirmations (block 207), patient treatment (block 208), and updating of patient records (block 209).

While various embodiments are targeted at metallic implants, other embodiments may be used with other materials that still provide conductivity for electrical current induced by AMF.

Example 5.21 The system according to example 5.2, wherein the therapeutic characteristic includes Tₘₐₓ=65° C., Δtₑₓₚ=5 min, t_{dose}=15 min.

Example 5.22 The system according to example 5.2, wherein the therapeutic characteristic includes Tₘₐₓ<80° C., Δtₑₓₚ between 2 and 7 min, t_{dose} between 5 and 60 min, and texp less than 10 seconds.

In some variations of example 5.22 tₑₓₚ is less than 50 ms. In some variations of example 5.22 texp is between 1 ms and 50 ms.

In some embodiments, these values are critical values that provide brief exposures to the surface of an implant with sufficient cool-down time in between exposures that results in a therapeutic dose capable of eradicating (i.e., significantly reducing) biofilm while protecting surrounding tissues from damage.

Another version of example 5.22 The system according to example 5.2, wherein the therapeutic characteristic includes Tₘₐₓ between 50 and 80° C., Δtₑₓₚ between 1 and 10 min, t_{dose} between 5 and 120 min, and texp less than 10 seconds.

Example 5.23 The system according to example 5.2, wherein the therapeutic characteristic includes Tₘₐₓ=65° C., Δtₑₓₚ=5 min, Nₑₓₚ=12, N_{dose}=2, Δt_{dose}=24 h, and tₑₓₚ less than 10 seconds.

Example 5.231 The system according to example 5.2, wherein the therapeutic characteristic includes Tₘₐₓ=between 55 and 75° C., Δtₑₓₚ=between 2 and 7 min, Nₑₓₚ=between 5 and 20, N_{dose}=between 1 and 5, Δt_{dose}=between 10 and 30 h, and tₑₓₚ between 2 and 10 seconds.

Example 5.24 The system according to example 5.2, wherein the therapeutic characteristic is configured to disrupt bacterial membranes of a biofilm included on the metallic implant.

The ability to adjust the one or more therapeutic characteristics unexpectedly provides the ability to reduce certain multidrug-resistant bacteria based on the mechanism of resistance.

Example 5.25 The system according to example 5.2, wherein the therapeutic characteristic includes Tₘₐₓ=65° C., Δtₑₓₚ=5 min, Nₑₓₚ=12, N_{dose}=2, Δt_{dose}=24 h, and tₑₓₚ less than 10 seconds.

Example 5.26 The system according to example 5.2, wherein the therapeutic characteristic includes Tₘₐₓ=between 45 and 85° C.

Example 5.27 The system according to example 5.2, wherein the therapeutic characteristic includes Δtₑₓₚ between 2 and 10 min.

Example 5.28 The system according to example 5.2, wherein the therapeutic characteristic includes Nₑₓₚ between 3 and 50.

Example 5.29 The system according to example 5.2, wherein the therapeutic characteristic includes N_{dose}=between 1 and 7.

Example 5.30 The system according to example 5.2, wherein the therapeutic characteristic includes Δt_{dose}=between 10 and 30 h.

Example 5.31 The system according to example 5.2, wherein the therapeutic characteristic includes tₑₓₚ between 1 and 15 seconds.

Example 5.32 The system according to example 5.2, wherein the therapeutic characteristic includes a frequency of less than 300 kHz. This helps reduce harm to tissue surrounding the implant. Other embodiments are between 175 and 225 kHz, or 150 and 25 kHz.

Another version of Example 5. The system of example 4, wherein: the at least one machine-readable medium comprises a first protocol configured for a first metallic implant and a second protocol configured for a second metallic implant; the first metallic implant has a first physical characteristic and the second metallic implant has a second physical characteristic that is unequal to the first physical characteristic; the first protocol includes a first magnitude of a therapeutic characteristic and the second protocol includes a second magnitude of the therapeutic characteristic that is unequal to the first magnitude of the therapeutic characteristic.

In an embodiment, the first physical characteristic concerns a first type of biofilm and the second physical characteristic concerns a second type of biofilm. For example, the first and second types of biofilm may concern first and second types of bacteria that are unequal to each other. The protocol may call for a larger maximum temperature for the first type of bacteria versus the second type of bacteria.

Another version of Example 5. The system of example 4, wherein: the at least one machine-readable medium comprises a first protocol configured for a first metallic implant and a second protocol configured for a second metallic implant; the first metallic implant has a first physical characteristic and the second metallic implant has a second physical characteristic that is unequal to the first physical characteristic; the first protocol includes a first therapeutic characteristic and the second protocol includes a second therapeutic characteristic that is unequal to the first therapeutic characteristic.

For example, one type of bacteria may be treated using pulse width modulation but no maximum temperature while another type of bacteria may be treated with a programmed maximum temperature.

Example 6. The system according to any of examples 5-5.2, wherein the first protocol includes a first period and the second protocol includes a second period that is unequal to the first period.

Example 7. The system according to any of examples 5-6, wherein the first protocol includes a first pulse width and the second protocol includes a second pulse width that is unequal to the first pulse width.

Example 8. The system according to any of examples 5-7, wherein: the first protocol includes a first duration of time to apply a plurality of pulses to the transmitter and the second protocol includes a second duration of time to apply a plurality of pulses to the transmitter; the first duration of time is unequal to the second duration of time.'

Example 9. The system according to any of examples 1-8, wherein the operations comprise communicating a plurality of AMF pulses to the metallic implant to raise a temperature on a surface of the metallic implant by less than 10 degrees Celsius in response to each of the plurality of pulses having a duty cycle of less than 1% and a period of between 1 ms and 60 seconds.

Example 10. The system according to any of examples 1-9, wherein the operations comprise communicating a plurality of AMF pulses to the metallic implant to induce a current on the surface of the metallic implant of between 50 and 3000 A/cm\hat{ }2 in response to each of the plurality of pulses having a duty cycle of less than 1% and a period of between 1 ms and 60 seconds.

Example 11. The system according to any of examples 1-10 comprising at least one sensor, wherein the operations comprise: sensing a parameter with the at least one sensor; changing at least one of the duty cycle or the period in response to sensing the parameter.

Example 11.1 The system according to example 11, wherein the operations comprise changing the therapeutic characteristic in response to sensing the parameter.

Example 12. The system according to any of example 11-11.1, wherein the parameter includes at least one of sound, temperature, resonance, energy, or combinations thereof.

For example, this may include sound or temperature in the immediate area of the implant. For example, see systems such as those described in U.S. Patent Application Publication Number 2019/0159725.

Further, sensing may cooperate with sensing embedded in or coupled to the implant. For example, if the implant itself has a built-in temperature monitor, such a monitor wirelessly (e.g., Bluetooth, etc.) communicates with the system. Thus, the system can sense the temperature near the device and modulate a therapeutic characteristic (e.g., duty cycle) to adjust the temperature to a target temperature, such as Tmax or a percentage thereof.

Example 21. A system comprising: at least one alternating magnetic field (AMF) transmitter configured to apply one or more AMF pulses to a metallic implant; at least one function generator; at least one processor; and at least one machine-readable medium having stored thereon data which, if used by the at least one processor, causes the at least one processor, the at least one function generator, and the at least one transmitter to perform operations comprising communicating a plurality of AMF pulses to the metallic implant; wherein the at least one machine-readable medium comprises a first protocol configured for a first metallic implant and a second protocol configured for a second metallic implant; the first metallic implant has a first magnitude of a physical characteristic and the second metallic implant has a second magnitude of the physical characteristic that is unequal to the first magnitude of the physical characteristic; the first protocol includes a first magnitude of a therapeutic characteristic and the second protocol includes a second magnitude of the therapeutic characteristic that is unequal to the first magnitude of the therapeutic characteristic.

Example 22. The system of example 21, wherein the physical characteristic includes one of density (kg/m\hat{ }3), electrical conductivity (S/m), relative permittivity, or thermal conductivity (W/(m·K)), specific heat (J/(kg·K)).

Example 23. The system according to any of examples 21-22, wherein the therapeutic characteristic includes one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

Example 24. The system according to any of examples 21-23, wherein the plurality of AMF pulses has a magnetic field no greater than 5 milliTesla (mT).

Example 25. The system according to any of examples 21-24, wherein each of the plurality of pulses has a pulse width of between 2 ms and 50 ms.

Example 26. The system according to any of examples 21-25, wherein the operations comprise communicating the plurality of AMF pulses to the metallic implant for a duration of at least 30 minutes.

Example 27. The system according to any of example 21-26, wherein the first protocol includes a first duty cycle and the second protocol includes a second duty cycle that is unequal to the first duty cycle.

Example 28. The system of example 27, wherein the first duty cycle is less than 1%.

Example 29. The system according to any of examples 21-28, wherein the first protocol includes a first period and the second protocol includes a second period that is unequal to the first period.

Example 30. The system of example 28, wherein the first period is between 1 ms and 60 seconds.

Example 31. The system according to any of examples 21-30, wherein the first protocol includes a first pulse width and the second protocol includes a second pulse width that is unequal to the first pulse width.

Example 32. The system according to any of examples 21-31, wherein: the first protocol includes a first duration of time to apply a plurality of pulses to the transmitter and the second protocol includes a second duration of time to apply a plurality of pulses to the transmitter; the first duration of time is unequal to the second duration of time.

Example 33. The system according to any of examples 21-32, wherein the operations comprise communicating a plurality of AMF pulses to the metallic implant to raise a temperature on a surface of the metallic implant by less than 10 degrees Celsius in response to each of the plurality of pulses having a duty cycle of less than 1% and a period of between 1 ms and 60 seconds.

Example 34. The system according to any of examples 21-33, wherein the operations comprise communicating a plurality of AMF pulses to the metallic implant to induce a current on the surface of the metallic implant of between 50 and 3000 A/cm\hat{ }2 in response to each of the plurality of pulses having a duty cycle of less than 1% and a period of between 1 ms and 60 seconds.

Example 35. The system according to any of examples 21-34 comprising at least one sensor, wherein the operations comprise: sensing a parameter with the at least one sensor; changing at least one of the duty cycle or the period in response to sensing the parameter.

Example 36. The system according to example 35, wherein the operations comprise changing the therapeutic characteristic in response to sensing the parameter.

Example 37. The system according to any of examples 35-36, wherein the parameter includes at least one of sound, temperature, resonance, energy, or combinations thereof.

Example 41. The at least one machine-readable medium according to any of examples 1-37.

For example, an embodiment includes software independent of AMF transmitters, function generators, computers, and the like.

Example 51. A method executed by at least one processor comprising: communicating a plurality of AMF pulses to the metallic implant in response to a user selecting one of first or second protocols via a user interface; wherein the first protocol is configured for a first metallic implant and the second protocol configured for a second metallic implant; wherein the first metallic implant has a first magnitude of a physical characteristic and the second metallic implant has a second magnitude of the physical characteristic that is unequal to the first magnitude of the physical characteristic; wherein the first protocol includes a first magnitude of a therapeutic characteristic and the second protocol includes a second magnitude of the therapeutic characteristic that is unequal to the first magnitude of the therapeutic characteristic.

Example 52. The method of example 51, wherein the physical characteristic includes one of density (kg/m\hat{ }3), electrical conductivity (S/m), relative permittivity, or thermal conductivity (W/(m·K)), specific heat (J/(kg·K)).

Example 53. The method according to any of examples 51-52, wherein the therapeutic characteristic includes one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

Example 54. The method according to any of examples 51-53, wherein the plurality of AMF pulses has a magnetic field no greater than 5 milliTesla (mT).

Example 55. The method according to any of examples 51-54, wherein each of the plurality of pulses has a pulse width of between 2 ms and 50 ms.

Example 56. The method according to any of examples 51-55, comprising communicating the plurality of AMF pulses to the metallic implant for a duration of at least 30 minutes.

Example 57. The method according to any of example 51-56, wherein the first protocol includes a first duty cycle and the second protocol includes a second duty cycle that is unequal to the first duty cycle.

Example 58. The method of example 57, wherein the first duty cycle is less than 1%.

Example 59. The method according to any of examples 51-58, wherein the first protocol includes a first period and the second protocol includes a second period that is unequal to the first period.

Example 60. The method of example 58, wherein the first period is between 1 ms and 60 seconds.

Example 61. The method according to any of examples 51-60, wherein the first protocol includes a first pulse width and the second protocol includes a second pulse width that is unequal to the first pulse width.

Example 62. The method according to any of examples 51-61, wherein: the first protocol includes a first duration of time to apply a plurality of pulses to the transmitter and the second protocol includes a second duration of time to apply a plurality of pulses to the transmitter; the first duration of time is unequal to the second duration of time.

Example 63. The method according to any of examples 51-62 comprising communicating a plurality of AMF pulses to the metallic implant to raise a temperature on a surface of the metallic implant by less than 10 degrees Celsius in response to each of the plurality of pulses having a duty cycle of less than 1% and a period of between 1 ms and 60 seconds.

Example 64. The method according to any of examples 51-63 comprising communicating a plurality of AMF pulses to the metallic implant to induce a current on the surface of the metallic implant of between 50 and 3000 A/cm\hat{ }2 in response to each of the plurality of pulses having a duty cycle of less than 1% and a period of between 1 ms and 60 seconds.

Example 65. The method according to any of examples 51-64 including: sensing a parameter with at least one sensor; changing at least one of the duty cycle or the period in response to sensing the parameter.

Example 66. The method according to example 65 comprising changing the therapeutic characteristic in response to sensing the parameter.

Example 67. The method according to any of examples 65-66, wherein the parameter includes at least one of sound, temperature, resonance, energy, or combinations thereof.

Example 71. A method comprising: using at least one alternating magnetic field (AMF) transmitter, at least one function generator, at least one processor, and at least one machine-readable medium having stored thereon data which, if used by the at least one processor, causes the at least one processor, the at least one function generator, and the at least one transmitter to perform operations comprising communicating a plurality of AMF pulses to the metallic implant, to communicate a plurality of AMF pulses to the metallic implant; wherein each of the plurality of AMF pulses has a duty cycle of less than 1% and a period of between 1 ms and 60 seconds.

Example 72. The method of example 71, wherein the plurality of AMF pulses has a magnetic field no greater than 5 milliTesla (mT).

Example 73. The method according to any of examples 71-7, wherein each of the plurality of pulses has a pulse width of between 2 ms and 50 ms.

Example 74. The method according to any of examples 1-3 comprising communicating the plurality of AMF pulses to the metallic implant for a duration of at least 30 minutes.

Example 75. The method according to any of examples 71-74 a user selecting at least one of first and second protocols, wherein: the at least one machine-readable medium comprises the first protocol configured for a first metallic implant and the second protocol configured for a second metallic implant; the first metallic implant has a first physical contour and the second metallic implant has a second physical contour that is unequal to the first physical contour; the first protocol includes a first duty cycle and the second protocol includes a second duty cycle that is unequal to the first duty cycle.

Another version of Example 75. The method of example 74, wherein: the at least one machine-readable medium comprises a first protocol configured for a first metallic implant and a second protocol configured for a second metallic implant; the first metallic implant has a first magnitude of a physical characteristic and the second metallic implant has a second magnitude of the physical characteristic that is unequal to the first magnitude of the physical characteristic; the first protocol includes a first duty cycle and the second protocol includes a second duty cycle that is unequal to the first duty cycle.

Another version of Example 75. The method of example 74, wherein: the at least one machine-readable medium comprises a first protocol configured for a first metallic implant and a second protocol configured for a second metallic implant; the first metallic implant has a first magnitude of a physical characteristic and the second metallic implant has a second magnitude of the physical characteristic that is unequal to the first magnitude of the physical characteristic; the first protocol includes a first magnitude of a therapeutic characteristic and the second protocol includes a second magnitude of the therapeutic characteristic that is unequal to the first magnitude of the therapeutic characteristic.

Example 75.1 The method of example 75, wherein the physical characteristic includes one of density (kg/m\hat{ }3), electrical conductivity (S/m), relative permittivity, or thermal conductivity (W/(m·K)), specific heat (J/(kg·K)).

Example 75.2 The method according to any of examples 71-75.1, wherein the therapeutic characteristic includes one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

Example 76. The method according to any of examples 75-75.2, wherein the first protocol includes a first period and the second protocol includes a second period that is unequal to the first period.

Example 77. The method according to any of examples 75-76, wherein the first protocol includes a first pulse width and the second protocol includes a second pulse width that is unequal to the first pulse width.

Example 78. The method according to any of examples 75-77, wherein: the first protocol includes a first duration of time to apply a plurality of pulses to the transmitter and the second protocol includes a second duration of time to apply a plurality of pulses to the transmitter; the first duration of time is unequal to the second duration of time.

Example 79. The method according to any of examples 71-78 comprising communicating a plurality of AMF pulses to the metallic implant to raise a temperature on a surface of the metallic implant by less than 10 degrees Celsius in response to each of the plurality of pulses having a duty cycle of less than 1% and a period of between 1 ms and 60 seconds.

Example 80. The method according to any of examples 71-79 comprising communicating a plurality of AMF pulses to the metallic implant to induce a current on the surface of the metallic implant of between 50 and 3000 A/cm\hat{ }2 in response to each of the plurality of pulses having a duty cycle of less than 1% and a period of between 1 ms and 60 seconds.

Example 81. The method according to any of examples 71-80 comprising: sensing a parameter with at least one sensor; changing at least one of the duty cycle or the period in response to sensing the parameter.

Example 81.1 The method according to examples 75.2 and 81 comprising changing the therapeutic characteristic in response to sensing the parameter.

Example 82. The method according to example 81, wherein the parameter includes at least one of sound, temperature, resonance, energy, or combinations thereof.

Example 83. The method according to any of examples 51 to 81 comprising administering a medication to the recipient of the AMF pulses within 1 week of that recipient of the AMF pulses receiving the AMF pulses.

However, in some embodiments no medication (e.g., antibiotic) is administered within 1 week of the patient receiving a dose of AMF pulses.

Applicant observed anomalous results where AMF exposures generating low temperatures (i.e., 50 degrees C. for 2 hours in a series of exposures) were toxic to biofilm when combined with antibiotics, even though equivalent exposures from conductive heating in a temperature-controlled water bath were ineffective. Applicants determined embodiments that produce electrical currents from AMF contribute to the sensitization of biofilm to antibiotics. Applicant evaluated short duration bursts with a low duty cycle that would generate high surface currents but would leave sufficient time in between bursts for heat to dissipate and not harm tissue. These provided unexpected therapeutic results. While initial thought might be that biofilm reduction is largely a function of temperature of the film, Applicant was able to determine that characteristics such as low pulse width, applied intermittently, when coupled with antibiotics were able to reduce biofilm without having to resort to high temperatures that can harm tissue surrounding the implant.

Thus, an unexpected result occurred. The expectation was that a low level of energy in conjunction with antibiotics would NOT reduce biofilm.

Example 84. The method according to any of examples 51 to 83 comprising sustain temperature on the metallic implant between 50-80° C. for more than 2 minutes.

Example 85. A method comprising: administering antibiotics to a patient; administering short duration AMF exposures repeatedly to a metallic implant within the patient with sufficient cool-down time in between exposures to allow for thermal doses that are therapeutic on the implant surface without a concomitant rise in tissue thermal dose.

Example 86. The method of example 85 including adjusting at least one AMF parameter configured to allow for thermal doses that are therapeutic on the implant surface without a concomitant rise in tissue thermal dose, wherein the at least one AMF parameter includes at least one of maximum temperature on the implant, duration of application of AMF impulses to the patient, and # of exposures per dose.

The ability to non-invasively induce low-temperature treatments while still generating significant electrical currents differentiates embodiments from conventional systems/methods. Also, because lower temperature treatments are desired, lower power amplifiers are needed. Such amplifiers are more affordable than larger amplifiers and should make the system more affordable to clinics of smaller size.

Embodiments may include a user interface. Such a user interface may include a touch screen. Such an embodiment may operate as a stand-alone instrument, without the need for any internet connection to provide treatment. However, a wireless connection may be used to download patient imaging data prior to treatment. The embodiment may be used in a clinical setting (e.g., outpatient or in an operating room). Orthopedists/orthopedic surgeons may use the system initially, but operation may be delegated to a technician under their oversight and direction. The technician may set-up the system (e.g., power-on the device, download appropriate patient records/images, and position the treatment transducer coil over or around the patient treatment area) and be present with the patient for the duration of the treatment.

The embodiment may provide, via logic, interruption of treatment if either a high-temperature signal is received from a safety sensor (e.g., acoustic sensor that monitors tissue adjacent implant to be treated), or if there is any abnormality detected in driving of the treatment transducer coil. Abnormalities Include: coil short circuit (overcurrent), coil open circuit (undercurrent), gantry arm movement, and the like.

An embodiment of a user interface may include patient data entry fields such as: Name, Patient ID Number, Date & Time, Menu to select implant, Image of implant w/ confirmation button. Thus, protocols mentioned herein may be selected based on the selection of a certain type of implant having various physical parameters. The user interface may display selected treatment parameters.

The user interface may include an area for positioning information (e.g., operator entering treatment transducer coil position information). The screen may include: an image of an implant, a “Positioned Correctly” button (for operator to confirm correct position of treatment transducer), and a “Start Treatment” button. The user interface may include an area for treatment information. The screen may include: a display of selected treatment parameters, a time display of treatment (progress bar), a “Stop Treatment” Button, and “Treatment Complete” indicators. The user interface may include an area for error information (e.g., treatment and other operations have been halted). The screen may indication: error: “Treatment Stopped” and “Cause of Error” (e.g., overtemperature, operator stopped before predetermined treatment time, high/low treatment power).

### REFERENCES

1. Arciola, C. R., Campoccia, D. & Montanaro, L. Implant infections: Adhesion, biofilm formation and immune evasion. Nat. Rev. Microbiol. 16, 397-409 (2018).
2. Darouiche, R. O. Treatment of Infections Associated with Surgical Implants. N. Engl. J. Med. 350,1422-1429 (2004).
3. Kurtz, S., Ong, K., Lau, E., Mowat, F. & Halpern, M. Projections of primary and revision hip and knee arthroplasty in the United States from 2005 to 2030. J. Bone Jt. Surg.-Ser. A 89,780-785 (2007).
4. Cui, Q., Mihalko, W. M., Shields, J. S., Ries, M. & Saleh, K. J. Antibiotic-impregnated cement spacers for the treatment of infection associated with total hip or knee arthroplasty. J. Bone Jt. Surg.-Ser. A 89,871-882 (2007).
5. O'Toole, P., Maltenfort, M. G., Chen, A. F. & Parvizi, J. Projected Increase in Periprosthetic Joint Infections Secondary to Rise in Diabetes and Obesity. J. Arthroplasty 31,7-10 (2016).
6. Beam, E. & Osmon, D. Prosthetic Joint Infection Update. Infect. Dis. Clin. North Am. 32,843-859 (2018).
7. Lee, J. et al. Risk factors for treatment failure in patients with prosthetic joint infections. J. Hosp. Infect. 75,273-276 (2010).
8. Hall-Stoodley, L., Costerton, J. W. & Stoodley, P. Bacterial biofilms: From the natural environment to infectious diseases. Nat. Rev. Microbiol. 2,95-108 (2004).
9. Tan, L. et al. Rapid Biofilm Eradication on Bone Implants Using Red Phosphorus and Near-Infrared Light. Adv. Mater. 30,1-10 (2018).
10. Jabbouri, S. & Sadovskaya, I. Characteristics of the biofilm matrix and its role as a possible target for the detection and eradication of Staphylococcus epidermidis associated with medical implant infections. FEMS Immunol. Med. Microbiol. 59,280-291 (2010).
11. Stewart, P. S. & Costerton, J. W. Antibiotic resistance of bacteria in biofilms. Lancet 358,135-138 (2001).
12. Bartlett, J. G. Treatment of infections associated with surgical implants. Infect. Dis. Clin. Pract. 12,258-259 (2004).
13. Zimmerli, W., Trampuz, A. & Ochsner, P. E. Prosthetic-Joint Infections. 1645-1654 (2018).
14. Jass, J. & Lappin-Scott, H. M. The efficacy of antibiotics enhanced by electrical currents against Pseudomonas aeruginosa biofilms. J. Antimicrob. Chemother. 38,987-1000 (1996).
15. Blenkinsopp, S. A., Khoury, A. E. & Costerton, J. W. Electrical enhancement of biocide efficacy against Pseudomonas aeruginosa biofilms. Appl. Environ. Microbiol. 58,3770-3773 (1992).
16. Van Der Borden, A. J., Van Der Werf, H., Van Der Mei, H. C. & Busscher, H. J. Electric current-induced detachment of Staphylococcus epidermidis biofilms from surgical stainless steel. Appl. Environ. Microbiol. 70,6871-6874 (2004).
17. Carmen, J. C. et al. Treatment of biofilm infections on implants with low-frequency ultrasound and antibiotics. Am. J. Infect. Control 33,78-82 (2005).
18. Ricker, E. B., Aljaafari, H. A. S., Bader, T. M. & Hundley, B. S. Thermal shock susceptibility and regrowth of Pseudomonas aeruginosa biofilms. Int. J. Hyperth. 0,168-176 (2018).
19. O'Toole, A., Ricker, E. B. & Nuxoll, E. Thermal mitigation of Pseudomonas aeruginosa biofilms. Biofouling 31,665-675 (2015).
20. Alumutairi, L., Yu, B., Filka, M., Nayfach, J. & Kim, M. H. Mild magnetic nanoparticle hyperthermia enhances the susceptibility of Staphylococcus aureus biofilm to antibiotics. Int. J. Hyperth. 37,66-75 (2020).
21. Gerdesmeyer, L. et al. Antibacterial effects of extracorporeal shock waves. Ultrasound Med. Biol. 31,115-119 (2005).
22. Chopra, R. et al. Employing high-frequency alternating magnetic fields for the non-invasive treatment of prosthetic joint infections. Sci. Rep. 7,1-14 (2017).
23. Ricker, E. B. & Nuxoll, E. Synergistic effects of heat and antibiotics on Pseudomonas aeruginosa biofilms. Biofouling 33,855-866 (2017).
24. Pijls, B. G., Sanders, I. M. J. G., Kuijper, E. J. & Nelissen, R. G. H. H. Synergy between induction heating, antibiotics, and N -acetylcysteine eradicates Staphylococcus aureus from biofilm. Int. J. Hyperth. 37,130-136 (2020).
25. Cheng, B. et al. Remote acoustic sensing as a safety mechanism during exposure of metal implants to alternating magnetic fields. PLoS One 13,1-20 (2018).
26. van Rhoon, G. C. et al. CEM43° C. thermal dose thresholds: a potential guide for magnetic resonance radiofrequency exposure levels? Eur. Radio!. 23, 2215-2227 (2013).
27. Chung, A. H., Jolesz, F. A. & Hynynen, K. Thermal dosimetry of a focused ultrasound beam in vivo by magnetic resonance imaging. Med. Phys. 26, 2017-2026 (1999).
28. Köhler, M. O. et al. Volumetric HIFU ablation under 3D guidance of rapid MRI thermometry. Med. Phys. 36,3521-3535 (2009).
29. Habash, M. B., Park, A. J., Vis, E. C., Harris, R. J. & Khursigara, C. M. Synergy of silver nanoparticles and aztreonam against Pseudomonas aeruginosa PAO1 Biofilms. Antimicrob. Agents Chemother. 58,5818-5830 (2014).
30. Berenbaum, M. C. A method for testing for synergy with any number of agents. J. Infect. Dis. 137,122-130 (1978).
31. den Hollander, J. G., Mouton, J. W. & Verbrugh, H. A. Use of pharmacodynamic parameters to predict efficacy of combination therapy by using fractional inhibitory concentration kinetics. Antimicrob. Agents Chemother. 42,744-748 (1998).
32. Soboh, F., Khoury, A. E., Zamboni, A. C., Davidson, D. & Mittelman, M. W. Effects of ciprofloxacin and protamine sulfate combinations against catheter-associated Pseudomonas aeruginosa biofilms. Antimicrob. Agents Chemother. 39, 1281-1286 (1995).
33. Reid, G. et al. Effects of ciprofloxacin, norfloxacin, and ofloxacin on in vitro adhesion and survival of Pseudomonas aeruginosa AK1 on urinary catheters. Antimicrob. Agents Chemother. 38,1490-1495 (1994).
34. Shelburne, S. A. et al. Whole-genome sequencing accurately identifies resistance to extended-spectrum β-lactams for major gram-negative bacterial pathogens. Clin. Infect. Dis. 65,738-745 (2017).
35. Pijls, B. G., Sanders, I. M. J. G., Kujiper, E. J. & Nelissen, R. G. H. H. Induction heating for eradicating Staphylococcus ep.
36. Roca, M., Castillo, M., Marti, P., Althaus, R. L. & Molina, M. P. Effect of heating on the stability of quinolones in milk. J. Agric. Food Chem. 58,5427-5431 (2010).
37. Samara, E. et al. Antibiotic stability over six weeks in aqueous solution at body temperature with and without heat treatment that mimics the curing of bone cement. Bone Jt. Res. 6,296-306 (2017).
38. Sapareto, S. A. & Dewey, W. C. Thermal dose determination in cancer therapy. Int. J. Radiat. Oncol. 10,787-800 (1984).
39. Mitchell, B. S. An Introduction To Materials Engineering and Science for Chemical and Materials Engineers. (John Wiley & Sons, Inc., 2004).
40. Munaweera, I. et al. Temperature-sensitive liposomal ciprofloxacin for the treatment of biofilm on infected metal implants using alternating magnetic fields. Int. J. Hyperth. 34,189-200 (2018).

### Supplemental References

1. den Hollander, J. G., Mouton, J. W. & Verbrugh, H. A. Use of Pharmacodynamic Parameters To Predict Efficacy of Combination Therapy by Using Fractional Inhibitory Concentration Kinetics. Antimicrob. Agents Chemother. 42, 744-748 (1998).
2. Berenbaum, M. C. A Method for Testing for Synergy with Any Number of Agents. J. Infect. Dis. 137, 122-130 (1978).
3. Habash, M. B., Park, A. J., Vis, E. C., Harris, R. J. & Khursigara, C. M. Synergy of Silver nanoparticles and aztreonam against pseudomonas aeruginosa PAO1 Biofilms. Antimicrob. Agents Chemother. 58, 5818-5830 (2014).
4. Dall, G. F. et al. Unexpected synergistic and antagonistic antibiotic activity against Staphylococcus biofilms. J. Antimicrob. Chemother. 73, 1830-1840 (2018).

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A system comprising:
at least one alternating magnetic field (AMF) transmitter configured to apply one or more AMF pulses to a metallic implant;
at least one function generator;
at least one processor; and
at least one machine-readable medium having stored thereon data which, if used by the at least one processor, causes the at least one processor, the at least one function generator, and the at least one transmitter to perform operations comprising communicating a plurality of AMF pulses to the metallic implant;
wherein each of the plurality of AMF pulses has a duty cycle of less than 1% and a period of between 1 ms and 60 seconds.

2. The system of claim 1, wherein the plurality of AMF pulses has a magnetic field no greater than 5 milliTesla (mT).

3. The system of claim 1, wherein each of the plurality of pulses has a pulse width of between 1 ms and 30 seconds.

4. The system of claim 3, wherein the operations comprise communicating the plurality of AMF pulses to the metallic implant for a duration of at least 30 minutes.

5. The system of claim 1, wherein:
the at least one machine-readable medium comprises a first protocol configured for a first metallic implant and a second protocol configured for a second metallic implant;
the first metallic implant has a first magnitude of a physical characteristic and the second metallic implant has a second magnitude of the physical characteristic that is unequal to the first magnitude of the physical characteristic;
the first protocol includes a first magnitude of a therapeutic characteristic and the second protocol includes a second magnitude of the therapeutic characteristic that is unequal to the first magnitude of the therapeutic characteristic.

6. The system of claim 5, wherein the physical characteristic includes at least one of density (kg/m\hat{ }3), electrical conductivity (S/m), relative permittivity, or thermal conductivity (W/(m·K)), specific heat (J/(kg·K)).

7. The system of claim 6, wherein the therapeutic characteristic includes at least one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

8. The system of claim 7, wherein the therapeutic characteristic includes a Tₘₐₓ between 50 and 80° C., a Δtₑₓₚ between 1 and 10 min, a t_{dose} between 5 and 120 min, and a tₑₓₚ less than 10 seconds.

9. The system of claim 8, wherein the therapeutic characteristic includes a Nₑₓₚ between 3 and 50, an N_{dose}=between 1 and 7, and a Δt_{dose}=between 10 and 30 h.

10. The system of claim 6, wherein:
the first protocol includes a first period and the second protocol includes a second period that is unequal to the first period;
the first protocol includes a first pulse width and the second protocol includes a second pulse width that is unequal to the first pulse width.

11. The system of claim 10, wherein:
the first protocol includes a first duration of time to apply a first plurality of pulses to the transmitter and the second protocol includes a second duration of time to apply a second plurality of pulses to the transmitter;
the first duration of time is unequal to the second duration of time.

12. The system of claims 1, wherein the operations comprise communicating the plurality of AMF pulses to the metallic implant to raise a temperature on a surface of the metallic implant by less than 10 degrees Celsius in response to each of the plurality of pulses having a duty cycle of less than 1% and a period of between 1 ms and 60 seconds.

13. The system of claim 1, wherein the operations comprise communicating the plurality of AMF pulses to the metallic implant to induce a current on the surface of the metallic implant of between 50 and 3000 A/cm\hat{ }2 in response to each of the plurality of pulses having a duty cycle of less than 1% and a period of between 1 ms and 60 seconds.

14. The system of claim 1 comprising at least one sensor, wherein the operations comprise:
sensing a parameter with the at least one sensor;
changing at least one of the duty cycle or the period in response to sensing the parameter.

15. The system of claim 14, wherein:
the operations comprise changing a therapeutic characteristic in response to sensing the parameter;
the therapeutic characteristic includes at least one of a total number of doses (N_{dose}), a length of exposure time (seconds) for each pulse (tₑₓₚ), a length of time between pulses of a dose (Δtₑₓₚ), a number of AMF pulses for each dose (Nₑₓₚ), a duration of time (hours) of each dose (dosing duration or t_{dose}), a fixed time interval (minutes) between two of the doses (Δt_{dose}) to allow the metallic implant to cool, a maximum target temperature (degrees Celsius) for the metallic implant (Tₘₐₓ).

16. A method comprising:
using at least one alternating magnetic field (AMF) transmitter, at least one function generator, and at least one processor to communicate a plurality of AMF pulses to the metallic implant;
wherein each of the plurality of AMF pulses has a duty cycle of less than 1% and a period of between 1 ms and 60 seconds.

17. The method of claim 16, wherein:
the plurality of AMF pulses has a magnetic field no greater than 5 milliTesla (mT);
each of the plurality of pulses has a pulse width of between 2 ms and 50 ms.

18. The method of claim 17 comprising communicating the plurality of AMF pulses to the metallic implant for a duration of at least 30 minutes.

19. A method comprising:
administering antibiotics to a patient having an implanted metallic implant;
administering alternating magnetic field (AMF) exposures repeatedly to the implant with cool-down time, which occurs between the exposures, that is configured to both: (a) allow for therapeutic thermal doses on a surface of the implant, and (b) avoid causing an excessive concomitant rise in tissue thermal dose;
wherein each of the exposures has a duty cycle of less than 1% and a period of between 1 ms and 60 seconds.

20. The method of claim 19 including adjusting at least one AMF parameter to modify the exposures to generate the cool-down time that is configured to configured to both: (a) allow for therapeutic thermal doses on a surface of the implant, and (b) avoid causing an excessive concomitant rise in tissue thermal dose, wherein the at least one AMF parameter includes at least one of maximum temperature on the implant, duration of application of AMF impulses to the patient, and number of exposures per dose of exposures.
